(19) United States
(12) Patent Application Publication
(10) Pub. No.: US 2024/0021284 A1
(43) Pub. Date: Jan. 18, 2024

(54) Location-Aided Glycemic Control

(71) Applicant: Dexcom, Inc., San Diego, CA (US)

(72) Inventors: Nathanael R. Paul, San Diego, CA (US); Apurv ULLAS Kamath, San Diego, CA (US)

(73) Assignee: Dexcom, Inc., San Diego, CA (US)

(21) Appl. No.: 18/220,565

(22) Filed: Jul. 11, 2023

Related U.S. Application Data
(60) Provisional application No. 63/389,491, filed on Jul. 15, 2022.

(51) Int. Cl. G16H 20/17; A61M 5/142; A61M 5/172

(52) U.S. Cl. CPC G16H 20/17; A61M 5/142; A61M 5/1723; A61M 2005/14208; A61M 2230/201

(57) ABSTRACT

Location-aided glycemic control is described. A glycemic control system obtains sensor data is from one or more sensors, and detects a location of a user based on the sensor data. The glycemic control system predicts an activity that the user will perform at the location based on the location of the user, and generates a recommendation to control a glycemic response of the user to the predicted activity. In one or more implementations the glycemic control system causes display of the recommendation to control the glycemic response of the user. In one or more implementations the recommendation corresponds to administering an amount of a medicament to the user to control the glycemic response to the predicted activity, and the glycemic control system communicates instructions to a medicament delivery system which causes the medicament delivery system to administer the amount of the medicament to the user.

### RELATED APPLICATION

This application claims priority under 35 U.S.C. § 119(e) to U.S. Provisional Patent Application No. 63/389,491, filed Jul. 15, 2022, and titled “Location-Aided Glycemic Control,” the entire disclosure of which is hereby incorporated by reference.

### BACKGROUND

Diabetes is a metabolic condition affecting hundreds of millions of people. For these people, monitoring blood glucose levels and regulating those levels to be within an acceptable range is important not only to mitigate long-term issues such as heart disease and vision loss, but also to avoid the effects of hyperglycemia and hypoglycemia. Maintaining blood glucose levels within an acceptable range can be challenging, as this level is almost constantly changing over time and in response to everyday events and activities, such as eating, exercising, sleeping, and stress.

### SUMMARY

To overcome these problems, location-aided glycemic control is leveraged. A glycemic control system obtains sensor data from one or more sensors, and detects a location of a user based on the sensor data. The glycemic control system predicts an activity that the user will perform based on the location of the user, and generates a recommendation to control a glycemic response of the user to the predicted activity. In one or more implementations, the glycemic control system causes display of the recommendation to control the glycemic response of the user. In one or more implementations, the recommendation corresponds to administering an amount of a medicament to the user to control the glycemic response to the predicted activity, and the glycemic control system communicates instructions to a medicament delivery system which causes the medicament delivery system to administer the amount of the medicament to the user.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ location-based glycemic control.

FIG. 2 depicts an example implementation of the analyte monitoring device of FIG. 1 in greater detail.

FIG. 3 depicts an example implementation of the medicament delivery system of FIG. 1 in greater detail.

FIG. 4 depicts an example of a system that receives and processes sensor data from various sources to generate recommendations for glycemic control based on a location of a user.

FIG. 5 depicts an example of mappings for location-aided glycemic control.

FIG. 6 depicts an example of a user interface displaying an activity notification.

FIG. 7 depicts an additional example of a user interface displaying an activity notification.

FIG. 8 depicts an additional example of a user interface displaying an activity notification.

FIG. 9 depicts an additional example of a user interface displaying an activity notification.

FIG. 10 depicts an example of a first combination of devices for implementing location-based glycemic control.

FIG. 11 depicts an example of a second combination of devices for implementing location-based glycemic control.

FIG. 12 depicts an example of a third combination of devices for implementing location-based glycemic control.

FIG. 13 depicts an example of a fourth combination of devices for implementing location-based glycemic control.

FIG. 14 depicts an example of a fifth combination of devices for implementing location-based glycemic control.

FIG. 15 depicts a procedure in an example implementation of location-aided glycemic control.

FIG. 16 illustrates an example of a system that includes an example of a computing device that is representative of one or more computing systems and/or devices that may implement the various techniques described herein.

### DETAILED DESCRIPTION

### Overview

Location-aided glycemic control is described. In accordance with the described techniques, a system for location-aided glycemic control is configured to detect, based on sensor data, a location of a person and then predict, based on the location of the person, an activity that the person will perform at the location. In some cases, the location of the person can be detected based on at least two different types of sensor data. By way of example, global positioning system (GPS) data obtained from a user's smartphone can be used to detect that the user is at a location corresponding to a “gym”, while a signal strength of a wireless network connection associated with the gym location may be used to confirm that the user is at the gym. As another example, the at least two different types of sensor data may be usable to distinguish between at least two candidate locations. Consider, for example, that the location corresponding to the gym is “next door” to a different location corresponding to a restaurant. In this example, first sensor data (e.g., GPS data) may be used to detect that the user is either at the gym or the restaurant, while second sensor data (e.g., wireless network signal strength) may be used to select one of these two locations in order to determine the user's current location.

Based on the detected location, the glycemic control system predicts an activity that the user will perform at the location. In some cases, this prediction is based on historical data indicating that the user previously performed the activity at the location or historical data indicating that other users have previously performed the activity at the location. Alternatively or additionally, the glycemic control system predicts the activity that the user will perform at the location based on a location type (or tag) associated with the location, e.g., different gyms can be associated with a gym location type (or tag) and different restaurants can be associated with a restaurant location type (or tag). Continuing with the examples from above, if the system detects that the person is at the gym, then the system can predict that the person will perform an activity associated with exercise at the gym location. In contrast, if the system detects that the user is at the restaurant, then the system can predict that the user will perform an activity associated with eating at the restaurant location.

Notably, the performance of different activities may change analyte levels in the person. For example, exercising may reduce glucose levels of the person, whereas eating may raise glucose levels of the person. For certain users, such as users with type II Diabetes, such activities may cause the person's glucose levels to exceed a target range that is associated with a dangerous medical condition. As an example, eating a meal with too many carbohydrates may cause the person's glucose levels to increase above a target range causing the person to experience a hyperglycemic event, whereas intense exercise may cause the person's glucose levels to decrease below the target range causing the person to experience a hypoglycemic event.

Thus, in order to maintain the user's glucose levels within the target range based on the performance of the activity at the detected location, the system generates a recommendation to control the glycemic response of the user to the predicted activity. In some cases, the recommendation is output to the user. For example, based on a prediction that the user will perform various exercise-related activities at the gym location, the system may output a notification or alert on the person's smartphone instructing the person to consume carbohydrates prior to exercising at the gym. Consuming carbohydrates before exercise may cause the person's glucose levels to rise prior to exercising such that the person's blood glucose levels will remain within the target range after a subsequent drop in glucose levels caused by the exercise.

In one or more implementations, the recommendation may correspond to medicament delivery. In this scenario, the system can control a medicament delivery system (e.g., an insulin pen or pump) to deliver medicament to the person based on the recommendation. For example, based on a prediction that the person will consume a meal at a restaurant, the system may determine a bolus dose of insulin to deliver to the person to prevent the person's glucose levels from exceeding the target range for hyperglycemia, and then communicate control signals to the medicament delivery system which cause the medicament delivery system to automatically deliver the bolus dose of insulin to the person.

In some aspects, the techniques described herein relate to a method including: obtaining sensor data from one or more sensors; detecting, based on the sensor data, a location of a user; predicting, based on the location of the user, an activity that the user will perform at the location; and generating a recommendation to control a glycemic response of the user to the predicted activity.

In some aspects, the techniques described herein relate to a method, wherein the generating the recommendation further includes: predicting the glycemic response of the user to the predicted activity; determining whether the glycemic response of the user to the predicted activity will cause an adverse health event; and responsive to determining that the glycemic response of the user to the predicted activity will cause the adverse health event, determining a mitigating therapy to prevent the adverse health event, wherein the recommendation includes the mitigating therapy.

In some aspects, the techniques described herein relate to a method, wherein the detecting the location of the user is based on at least two different types of sensor data.

In some aspects, the techniques described herein relate to a method, wherein the detecting the location of the user includes detecting the location of the user based on first sensor data, and confirming the location of the user based on second sensor data.

In some aspects, the techniques described herein relate to a method, wherein the detecting the location of the user includes: detecting at least two candidate locations of the user based on first sensor data; and selecting one of the at least two candidate locations as the location of the user based on second sensor data.

In some aspects, the techniques described herein relate to a method, wherein the recommendation includes administering an amount of a medicament to the user to control the glycemic response of the user to the predicted activity.

In some aspects, the techniques described herein relate to a method, further including communicating instructions to a medicament delivery system, the instructions causing the medicament delivery system to administer the amount of the medicament to the user.

In some aspects, the techniques described herein relate to a method, wherein the medicament includes insulin.

In some aspects, the techniques described herein relate to a method, wherein the predicting the activity includes predicting, based on the location, a state of the activity, the state including a pre-activity state, an activity state, or a post-activity state, wherein the recommendation is based on the state of the activity.

In some aspects, the techniques described herein relate to a method, further including: receiving additional sensor data; determining, based on the additional sensor data, an additional location of the user; predicting, based on the additional location of the user, that the user is a different activity state; and generating an additional recommendation based on the different activity state.

In some aspects, the techniques described herein relate to a method, further including causing display of the recommendation to control the glycemic response of the user on a computing device.

In some aspects, the techniques described herein relate to a method, wherein the predicted activity includes exercising, eating, or sleeping.

In some aspects, the techniques described herein relate to a system including: one or more sensors to obtain sensor data associated with a user; a medicament delivery system to administer a medicament to the user; and at least a memory and a processor to perform operations including: detecting, based on the sensor data, a location of the user; predicting, based on the location of the user, an activity that the user will perform at the location; determining an amount of the medicament to administer to the user to control a glycemic response of the user to the predicted activity; and communicating instructions to the medicament delivery system, the instructions causing the medicament delivery system to deliver the amount of the medicament to the user.

In some aspects, the techniques described herein relate to a system, further including an analyte monitoring device to obtain analyte data of the user, wherein the analyte monitoring device and the medicament delivery system are connected in a closed loop.

In some aspects, the techniques described herein relate to a system, wherein the amount of the medicament is determined based at least in part on analyte data of the user obtained from the analyte monitoring device.

In some aspects, the techniques described herein relate to a system, wherein the instructions cause the medicament delivery system to deliver the amount of the medicament to the user without user input.

In some aspects, the techniques described herein relate to a system, wherein the medicament includes insulin, and wherein the medicament delivery system includes an insulin pump.

In some aspects, the techniques described herein relate to a method including: detecting, based on at least two different types of sensor data, a location of a user; predicting, based on the location of the user, an activity that the user will perform at the location; predicting a glycemic response of the user to the predicted activity; determining whether the glycemic response of the user to the predicted activity will cause an adverse health event; and responsive to determining that the glycemic response of the user to the predicted activity will cause the adverse health event, communicating instructions to an insulin pump to cause the insulin pump to control the glycemic response of the user by administering insulin to the user.

In some aspects, the techniques described herein relate to a method, further including obtaining glucose measurements of the user from a glucose monitoring device worn by the user, wherein the predicting the glycemic response further includes predicting the glycemic response of the user to the predicted activity based at least in part on the glucose measurements.

In some aspects, the techniques described herein relate to a method, wherein the glucose monitoring device and the insulin pump are connected in a closed loop.

In the following discussion, an exemplary environment is first described that may employ the techniques described herein. Examples of implementation details and procedures are then described which may be performed in the exemplary environment as well as other environments. Performance of the exemplary procedures is not limited to the exemplary environment and the exemplary environment is not limited to performance of the exemplary procedures.

### Example of an Environment

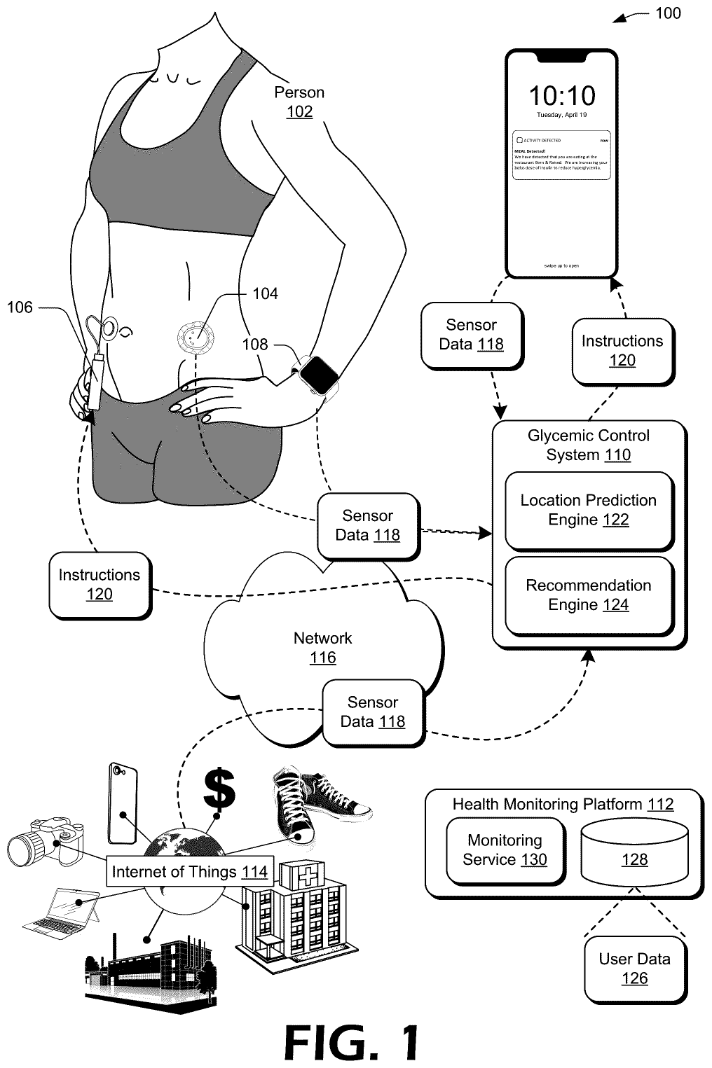

FIG. 1 is an illustration of an environment 100 in an example implementation that is operable to employ location-based glycemic control. The illustrated environment 100 includes person 102, who is depicted wearing analyte monitoring device 104 and medicament delivery system 106. The illustrated environment 100 also includes example computing devices 108, glycemic control system 110, health monitoring platform 112, and Internet of Things (IoT 114). The analyte monitoring device 104, the medicament delivery system 106, the example computing devices 108, the glycemic control system 110, the health monitoring platform 112, and the IoT 114 are communicably coupled, including via network 116.

The analyte monitoring device 104, the medicament delivery system 106, and one or more computing devices 108 (associated with the person 102) may be communicably coupled in various ways, such as by using one or more wireless communication protocols or techniques. By way of example, the analyte monitoring device 104, the medicament delivery system 106, and one or more computing devices 108 may communicate with one another using one or more of radio, cellular, Wi-Fi, Bluetooth (e.g., Bluetooth Low Energy links), near-field communication (NFC), and 5G, to name just a few.

Through such communicative couplings, the analyte monitoring device 104, the medicament delivery system 106, and one or more of the computing devices 108 may form a closed-loop system in one or more implementations. When implemented as a closed-loop system, the combination of devices is configured to provide location-based glycemic control in a way that eliminates or reduces user interaction involved with mitigating adverse health events (e.g., glycemic events). Instead, the devices process various information and make various predictions and determinations, such as about a user's location, activities the user is performing or will perform based on the location, glycemic responses to those activities, and therapies needed (if any) to mitigate those glycemic responses. In a closed-loop system, the devices can then provide the determined therapy without user interaction, e.g., the medicament delivery system 106 controlled to administer medicament to the person 102.

In one or more implementations, the analyte monitoring device 104 is a wearable, such that it is worn by the person 102 while the device performs various operations. Additionally or alternatively, the analyte monitoring device 104 performs one or more operations before or after being worn by the person 102. Broadly, the analyte monitoring device 104 is configured to provide measurements of an analyte of the person 102, e.g., the person 102's glucose. For instance, the analyte monitoring device 104 may be configured with an analyte sensor that detects one or more signals indicative of the analyte in the person 102 and enables generation of analyte measurements or estimations (e.g., estimated glucose values). Those analyte measurements (e.g., glucose measurements) may correspond to or otherwise be packaged for communication to one or more of the computing devices 108 or the medicament delivery system 106 as analyte data, which is one example of sensor data 118.

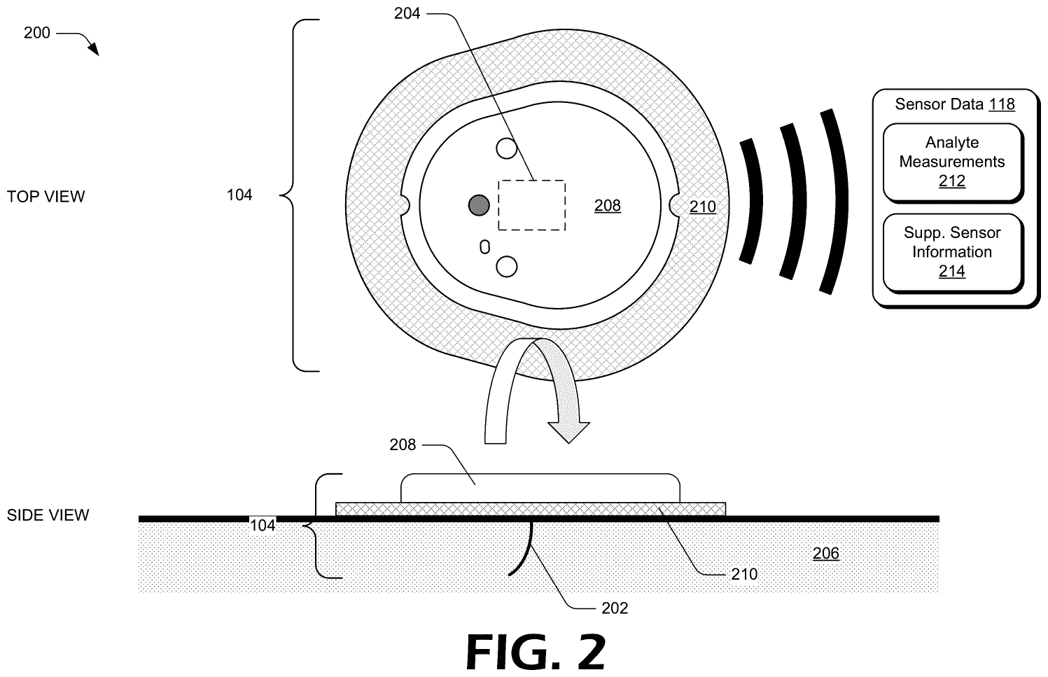

In at least one implementation, the analyte monitoring device 104 is a glucose monitoring system. As an example, the analyte monitoring device 104 may be configured as a continuous glucose monitoring (“CGM”) system, e.g., a wearable CGM system. As used herein, the term “continuous” when used in connection with analyte monitoring may refer to an ability of a device to produce measurements substantially continuously, such that the device may be configured to produce the analyte measurements at regular or irregular intervals of time (e.g., approximately every hour, approximately every 30 minutes, approximately every 5 minutes, and so forth), responsive to establishing a communicative coupling with a different device (e.g., when a computing device 108 establishes a wireless connection with the analyte monitoring device 104 to retrieve one or more of the measurements), and so forth. In other implementations, however, the glucose monitoring device may not be “continuous”, and instead provides glycose measurements when requested. This functionality along with further aspects of the configuration of the analyte monitoring device 104 are discussed in more detail in relation to FIG. 2.

In addition to producing sensor data 118 (including analyte data indicative of measurements of the analyte in the person 102), the analyte monitoring device 104 also transmits the produced sensor data 118, e.g., to the computing device 108. The analyte monitoring device 104 may communicate the data in real-time, e.g., as it is produced using an analyte sensor or other sensors. Alternatively or in addition, the analyte monitoring device 104 may communicate the data to a computing device 108 at predefined intervals of time. For example, the analyte monitoring device 104 may be configured to communicate the sensor data 118 to the computing device 108 approximately every five minutes. Certainly, an interval at which the sensor data 118 is communicated by the analyte monitoring device 104 may be different from the examples above without departing from the spirit or scope of the described techniques. The data may be communicated by the analyte monitoring device 104 to a computing device 108 according to other bases in accordance with the described techniques.

Additionally, the computing device 108 may maintain the sensor data 118 at least temporarily, e.g., in a storage device (not shown) of the computing device 108. The sensor data 118 may also be maintained in such storage of the computing device 108 or storage of a different device along with other associated data, such as with corresponding timestamps and/or identifiers of data packets in which communicated, to name just a few.

As depicted, the described systems may include one or more computing devices 108 in accordance with the described techniques. In one or more scenarios, for instance, the described techniques may be implemented using a computing device 108, such as a mobile phone. Alternatively, the described techniques are implementable using multiple computing devices 108, which in at least one variation may include both a wearable device (e.g., a smart watch, mouthguard, contact lenses, smart glasses, chest strap, ear buds, or headphones, to name just a few) and a mobile phone. In such scenarios, both of these devices may be capable of performing at least some of the same operations, such as to receive the sensor data 118 from the analyte monitoring device 104 (and from other sources), generate sensor data 118 using onboard or associated sensors, communicate data via the network 116 to the glycemic control system 110 and/or the health monitoring platform 112, display information related to the data, display information related to recommendations produced by the glycemic control system 110 and/or the health monitoring platform 112, facilitate control of other devices (e.g., controlling the medicament delivery system 106), and so forth. Alternatively or in addition, different devices may have different capabilities that other devices do not have or that are limited to specified devices through computing instructions.

The glycemic control system 110 causes therapy to be administered or recommended based on determinations made by processing the sensor data 118 and by providing instructions 120, such as by providing the instructions 120 to the medicament delivery system 106, the computing device 108, and/or to another device (e.g., an additional medicament delivery system). In accordance with the described techniques, the glycemic control system 110 makes those determinations and causes therapy to be administered or recommended, at least in part, by leveraging one or more of the computing devices 108, the analyte monitoring device 104, the medicament delivery system 106, the glycemic control system 110, and the IoT 114.

In the illustrated example, the glycemic control system 110 is depicted including location prediction engine 122 and recommendation engine 124. It is to be appreciated that the glycemic control system 110 may include more, fewer, or different components without departing from the spirit or scope of the described techniques. Although depicted as being separate from the analyte monitoring device 104, the medicament delivery system 106, the computing devices 108, and the health monitoring platform 112, in one or more implementations, at least a portion of the glycemic control system 110 is implemented at one or more of those entities. Additionally, various portions of the glycemic control system 110 are implementable at or otherwise using different combinations of the analyte monitoring device 104, the medicament delivery system 106, the computing devices 108, and the health monitoring platform 112 in accordance with the described techniques.

As discussed above and below, the glycemic control system 110 obtains the sensor data 118 from various sources. For example, the glycemic control system 110 obtains the sensor data 118 from one or more of the analyte monitoring device 104, the medicament delivery system 106, the computing devices 108, the health monitoring platform 112, or the IoT 114. Examples of the sensor data 118 include, but are not limited to, global positioning system (GPS) data, Wi-Fi information (e.g., service set identifiers (SSIDs) of available networks, networks to which the computing device 108 is connected, and/or networks to which the computing device 108 previously connected), Bluetooth Low Energy (BLE) information, data produced using a cellular antenna (e.g., long term evolution (LTE)), microphone data (e.g., sound data), accelerometer data, gyroscope data, magnetometer data, barometer data, ambient or internal temperature data (e.g., produced using temperature sensors), light data (e.g., captured using a device's camera), heart rate data (e.g., produced by a smartphone and/or a smart watch), proximity data (e.g., between devices), humidity data, analyte data, and medicament data, to name just a few. It is to be appreciated that this list is not exhaustive, and that the glycemic control system 110 may receive various other data (some of which is discussed above and below), without departing from the spirit or scope of the described techniques.

Based on the sensor data 118, the location prediction engine 122 detects a location of a user, e.g., the person 102 wearing the analyte monitoring device 104. In one or more implementations, the location prediction engine 122 detects the location of the user based on at least two different types of the sensor data 118. For instance, the location prediction engine 122 detects the location of the user based on both GPS data and Wi-Fi information (e.g., an SSID to which the computing device 108 of the user is connected) or based on Wi-Fi information and sound data. In one or more implementations, the location prediction engine 122 uses the first sensor data to detect a location of the user and the second sensor data to confirm the location of the user. Alternatively or in addition, the location prediction engine 122 uses the two types of sensor data to distinguish which of multiple candidate locations corresponds to the actual physical location of the user. For instance, the location prediction engine 122 detects at least two candidate locations of the user based on the first sensor data. Based on the second sensor data, the location prediction engine 122 selects one of the at least two candidate locations as the location of the user (or eliminates all but one of the candidate locations).

The recommendation engine 124 predicts an activity that the user will perform at the location. Examples of activities include eating, exercising, and sleeping. Certainly, the system may predict other activities without departing from the spirit or scope of the described techniques. The system then uses the activity the user is predicted to perform to generate one or more recommendations for controlling a glycemic response of the user, e.g., the person 102's glycemic response to the predicted activity. For example, the recommendation engine 124 generates recommendations for one or more therapies to mitigate potential adverse effects due to the predicted activity. Examples of adverse effects include at least glucose excursions from a safe glucose range, such as hyperglycemia or hypoglycemia.

As discussed above and below, the recommendation engine 124 may also use an “activity state” to generate such recommendations. This is because a therapy recommended prior to an activity may differ from therapies recommended during the activity or after the activity. By way of example, a therapy recommended prior to eating a meal may differ from therapies recommended while eating the meal and after eating the meal. In at least one implementation, activity states may include a pre-activity state, an activity state (e.g., during the activity), and a post-activity state. Activity states may be defined in other ways in variations, e.g., based on patterns identified in analyte data.

In one or more implementations, the glycemic control system 110 provides the instructions 120 to notify the user about a recommended therapy. For instance, the glycemic control system 110 provides the instructions 120 to the computing device 108 to cause the computing device 108 to display or otherwise output the recommendation (or information indicative of the recommendation). Alternatively or additionally, the glycemic control system 110 provides the instructions 120 to control a device (e.g., the medicament delivery system 106) to provide the recommended therapy. For instance, the glycemic control system 110 provides the instructions 120 to the medicament delivery system 106, and the instructions cause the medicament delivery system 106 to administer an amount of medicament to the person 102.

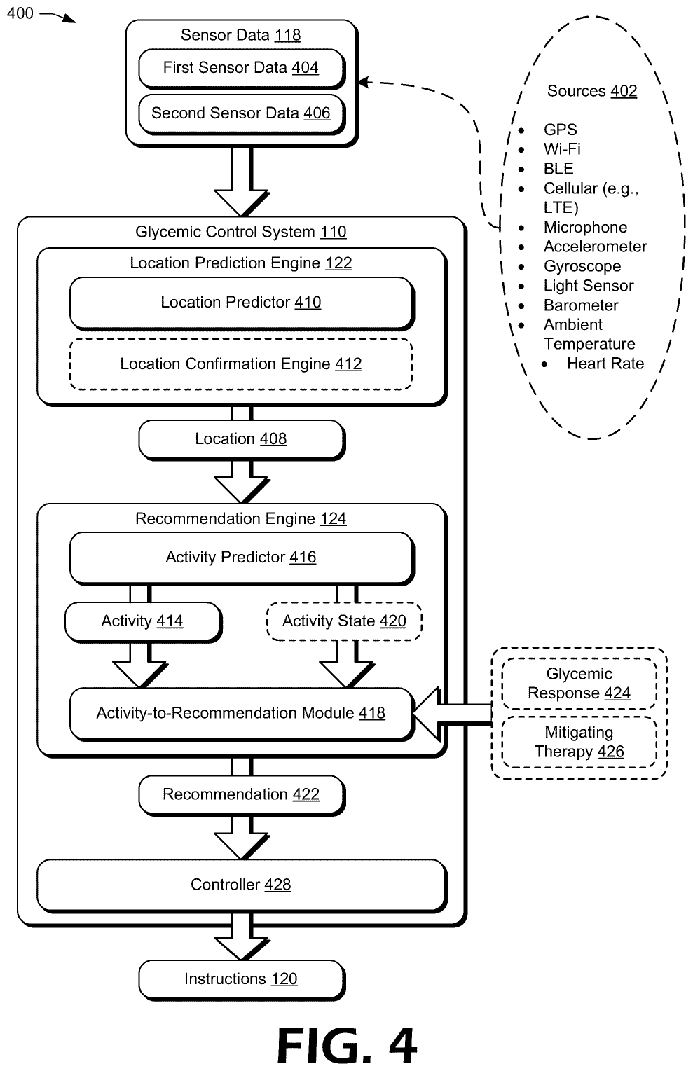

In one or more implementations, the instructions cause the medicament delivery system 106 to administer the medicament (e.g., a dose) to the person 102 automatically, without user input. In such implementations, the devices are operatively connected in a closed loop. In other implementations, the instructions instruct the medicament delivery system 106 to administer the medicament (e.g., a dose) to the person 102, but the system prevents the medicament from being delivered until a validation input is received from the user, e.g., an input indicating that the user allows the medicament to be delivered. Alternatively or additionally, the instructions instruct the medicament delivery system 106 to administer a dose of the medicament, and the medicament delivery system 106 requires user interaction to transfer the dose into the body of the person 102, e.g., a user is required to position the medicament delivery system 106 against the person 102's body and actuate the system (e.g., press a button) to administer the medicament. One example of the glycemic control system 110 is discussed in more detail below in relation to FIG. 4.

As noted above, the glycemic control system 110 uses sensor data 118 obtained via the IoT 114 in one or more implementations. It is to be appreciated that the IoT 114 represents various sources capable of providing data that describes the person 102 and/or the person 102's activity, such as the person 102's activity as a user of one or more service providers or the person 102's activity within the real world, e.g., at home, in an automobile, at work, in a gym, in a restaurant, or in other stores, to name just a few. By way of example, the IoT 114 may include various devices of the user, e.g., mobile phones, wearable devices, cameras, laptops, and so forth. To this end, the IoT 114 may provide information about interaction of the user with those various devices, e.g., location of those devices, environmental and/or physical conditions at locations where the devices are placed, interaction with web-based applications supported by the devices, interaction with health applications supported by the devices, photos taken, communications with other users, online behavior, and so forth. The IoT 114 may also include various other real-world articles (e.g., shoes, clothing, sporting equipment, appliances, smart-home devices, automobiles, etc.) configured with sensors to provide information describing behavior, such as steps taken, force of a foot striking the ground, length of stride, temperature of a user (and other physiological measurements), temperature of a user's surroundings, types of food stored in a refrigerator, types of food removed from a refrigerator, driving habits, images of the user at different times of the day, and so forth. Such other real-world articles can also provide sensor data 118 describing location of those articles, user interaction with those articles, environmental and/or physical conditions at locations where the articles are placed, and various other conditions.

In variations, the IoT 114 may also include third parties to the health monitoring platform 112, such as medical providers (e.g., a medical provider of the person 102) and manufacturers (e.g., manufacturers of the analyte monitoring device 104, the medicament delivery system 106, or one or more of the computing devices 108) capable of providing medical and manufacturing data, respectively, that can be leveraged by the glycemic control system 110. Certainly, the IoT 114 may include devices and sensors capable of providing a wealth of data used to determine a location and/or activities of the person 102 without departing from the spirit or scope of the described techniques.

In one or more implementations, the glycemic control system 110 also leverages resources of the health monitoring platform 112 in connection with location-aided glycemic control. For instance, the health monitoring platform 112 may be configured to store data, such as the sensor data 118 (e.g., analyte measurements, data produced by other sensors, and data produced based on determinations made using the analyte measurements and/or data produced by other sensors), the instructions 120, user profile data associated with a user (e.g., the person 102), and/or user profile data associated with one or more other users of a user population (not shown). The environment 100 includes user data 126, which represents a variety of data obtained and stored by the health monitoring platform 112 about one or more of those users. Although stored about one or more users, the user data 126 may be obfuscated using one or more techniques, such that personally identifying information of the users associated with the data can be kept anonymous in various scenarios of using the data. In the illustrated environment 100, the user data 126 is shown stored in the storage device 128. The storage device 128 may represent one or more databases or other storage included as part of or otherwise accessible to the health monitoring platform 112. In accordance with the described techniques, the storage device 128 is capable of storing the user data 126 and various other data.

By way of example, the user data 126 may include any combination of the data discussed just above as well as other data discussed above and below. For instance, the user data 126 may include historical data associated with the one or more users, such as historical sensor data 118, determinations made based on the historical sensor data 118, user inputs received, and so on. In at least one implementation, such historical data may describe conditions of the users and/or behaviors of the users, including, for instance, activities previously performed by a user at a location, activities previously performed by other users at the location, and attributes of the performed activities, e.g., an activity time, a location type, physiological measurements during the activity, etc.

In one or more implementations, the health monitoring platform 112 includes a monitoring service 130. The monitoring service 130 may be used separately or in connection with the glycemic control system 110. By way of example, the monitoring service 130 may provide one or more web-based health-related services to users via the network 116 and their computing devices 108, e.g., mobile applications. The health monitoring platform 112 may include or have access to various computing resources, such as processing, storage, and virtual resources. Those resources may be useable, for example, to train, maintain, and/or deploy algorithms (e.g., machine learning algorithms), which may generate predictions associated with health monitoring by using the wealth of data collected about the person 102 and users of a user population. Thus, the monitoring service 130 may leverage those resources to execute algorithms and to implement other functionality in order to deliver web-based services to users via their devices. Notably, one or more such algorithms or functionalities may require an amount of computing resources that exceeds the resources of typical, personal computing devices, e.g., mobile phones, laptops, tablet devices, and wearables, to name just a few. Nonetheless, the health monitoring platform 112 may include or otherwise have access to at least a threshold amount of resources needed to operate such algorithms and provide such functionality, e.g., cloud storage, server devices, virtualized resources, and so forth. The health monitoring platform 112 may include a variety of resources that the computing devices 108 can leverage via the monitoring service 130 in order to provide user interfaces or generate data for location-aided glycemic control. In the context of measuring an analyte, e.g., glucose continuously, and obtaining analyte data describing such measurements, consider the following discussion of FIG. 2.

FIG. 2 depicts an example 200 of an implementation of the analyte monitoring device 104 of FIG. 1 in greater detail. In particular, the illustrated example 200 includes a top view and a corresponding side view of the analyte monitoring device 104. It is to be appreciated that the analyte monitoring device 104 may vary in implementation from the following discussion in various ways without departing from the spirit or scope of the described techniques.

In this example 200, the analyte monitoring device 104 is illustrated to include an analyte sensor 202 (e.g., a glucose sensor) and a sensor module 204. Here, the analyte sensor 202 is depicted in the side view having been inserted subcutaneously into skin 206, e.g., of the person 102. The sensor module 204 is approximated in the top view as a dashed rectangle. The analyte monitoring device 104 also includes a transmitter 208 in the illustrated example 200. Use of the dashed rectangle for the sensor module 204 indicates that it may be housed or otherwise implemented within a housing of the transmitter 208. Antennae and/or other hardware used to enable the transmitter 208 to produce signals for communicating data, e.g., over a wireless connection to the medicament delivery system 106 and/or the computing device 108, may also be housed or otherwise implemented within the housing of the transmitter 208. In this example 200, the analyte monitoring device 104 further includes adhesive pad 210.

In operation, the analyte sensor 202 and the adhesive pad 210 may be assembled to form an application assembly, where the application assembly is configured to be applied to the skin 206 so that the analyte sensor 202 is subcutaneously inserted as depicted. In such scenarios, the transmitter 208 may be attached to the assembly after application to the skin 206 via an attachment mechanism (not shown). Alternatively, the transmitter 208 may be incorporated as part of the application assembly, such that the analyte sensor 202, the adhesive pad 210, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, user-initiated application of the analyte monitoring device 104 with a sensor applicator is nearly painless and does not require the withdrawal of blood. Moreover, the automatic sensor applicator generally enables the person 102 to embed the analyte sensor 202 subcutaneously into the skin 206 without the assistance of a clinician or healthcare provider.

The analyte monitoring device 104 may also be removed by peeling the adhesive pad 210 from the skin 206. It is to be appreciated that the analyte monitoring device 104 and its various components as illustrated are simply one example form factor, and the analyte monitoring device 104 and its components may have different form factors without departing from the spirit or scope of the described techniques.

In operation, the analyte sensor 202 is communicably coupled to the sensor module 204 via at least one communication channel which can be a wireless connection or a wired connection. Communications from the analyte sensor 202 to the sensor module 204 or from the sensor module 204 to the analyte sensor 202 can be implemented actively or passively and these communications can be continuous (e.g., analog) or discrete (e.g., digital).

The analyte sensor 202 may be a device, a molecule, and/or a chemical which changes or causes a change in response to an event which is at least partially independent of the analyte sensor 202. The sensor module 204 is implemented to receive indications of changes to the analyte sensor 202 or caused by the analyte sensor 202. For example, the analyte sensor 202 can include glucose oxidase which reacts with glucose and oxygen to form hydrogen peroxide that is electrochemically detectable by the sensor module 204 which may include an electrode. In this example, the analyte sensor 202 may be configured as or include a glucose sensor configured to detect analytes in blood or interstitial fluid that are indicative of glucose level using one or more measurement techniques. In one or more implementations, the analyte sensor 202 may also be configured to detect analytes in the blood or the interstitial fluid that are indicative of other markers, such as lactate levels, ketones, or ionic potassium, which may improve accuracy in identifying or predicting glucose-based events (e.g., hyperglycemia or hypoglycemia). Additionally or alternatively, the analyte monitoring device 104 may include additional sensors and/or architectures to the analyte sensor 202 to detect those analytes indicative of the other markers.

In another example, the analyte sensor 202 (or an additional sensor of the analyte monitoring device 104—not shown) can include a first and second electrical conductor and the sensor module 204 can electrically detect changes in electric potential across the first and second electrical conductor of the analyte sensor 202. In this example, the sensor module 204 and the analyte sensor 202 are configured as a thermocouple such that the changes in electric potential correspond to temperature changes. In some examples, the sensor module 204 and the analyte sensor 202 are configured to detect a single analyte, e.g., glucose. In other examples, the sensor module 204 and the analyte sensor 202 are configured to use diverse sensing modes to detect multiple analytes, e.g., ionic sodium, ionic potassium, carbon dioxide, and glucose. Alternatively or additionally, the analyte monitoring device 104 includes multiple sensors to detect not only one or more analytes (e.g., ionic sodium, ionic potassium, carbon dioxide, glucose, and insulin) but also one or more environmental conditions (e.g., temperature, moisture, movement). Thus, the sensor module 204 and the analyte sensor 202 (as well as any additional sensors) may detect the presence of one or more analytes, the absence of one or more analytes, and/or changes in one or more environmental conditions. As noted above, the analyte monitoring device 104 may be configured to produce data describing a single analyte (e.g., glucose) or multiple analytes.

In one or more implementations, the sensor module 204 may include a processor and memory (not shown). The sensor module 204, by leveraging the processor, may generate analyte measurements 212 based on the communications with the analyte sensor 202 that are indicative of the above-discussed changes. Based on the above-noted communications from the analyte sensor 202, the sensor module 204 is further configured to generate communicable packages of data that include at least one analyte measurement 212. In this example 200, the sensor data 118 represents these packages of data. Additionally or alternatively, the sensor module 204 may configure the sensor data 118 to include additional data, including, by way of example, supplemental sensor information 214. The supplemental sensor information 214 may include a sensor identifier, a sensor status, temperatures that correspond to the analyte measurements 212, measurements of other analytes that correspond to the analyte measurements 212, and so forth. It is to be appreciated that supplemental sensor information 214 may include a variety of data that supplements at least one analyte measurement 212 without departing from the spirit or scope of the described techniques.

In implementations where the analyte monitoring device 104 is configured for wireless transmission, the transmitter 208 may transmit the sensor data 118 as a stream of data to a computing device. Alternatively or additionally, the sensor module 204 may buffer the analyte measurements 212 and/or the supplemental sensor information 214 (e.g., in memory of the sensor module 204 and/or other physical computer-readable storage media of the analyte monitoring device 104) and cause the transmitter 208 to transmit the buffered sensor data 118 later at various regular or irregular intervals, e.g., time intervals (approximately every second, approximately every thirty seconds, approximately every minute, approximately every five minutes, approximately every hour, and so on), storage intervals (when the buffered analyte measurements 212 and/or supplemental sensor information 214 reach a threshold amount of data or a number of measurements), and so forth. It should be appreciated that in implementations the analyte monitoring device 104 can vary in numerous ways from the example described above without departing from the spirit or scope of the described techniques.

Having discussed one example of an analyte monitoring device, consider now the following discussion of one example medicament delivery system.

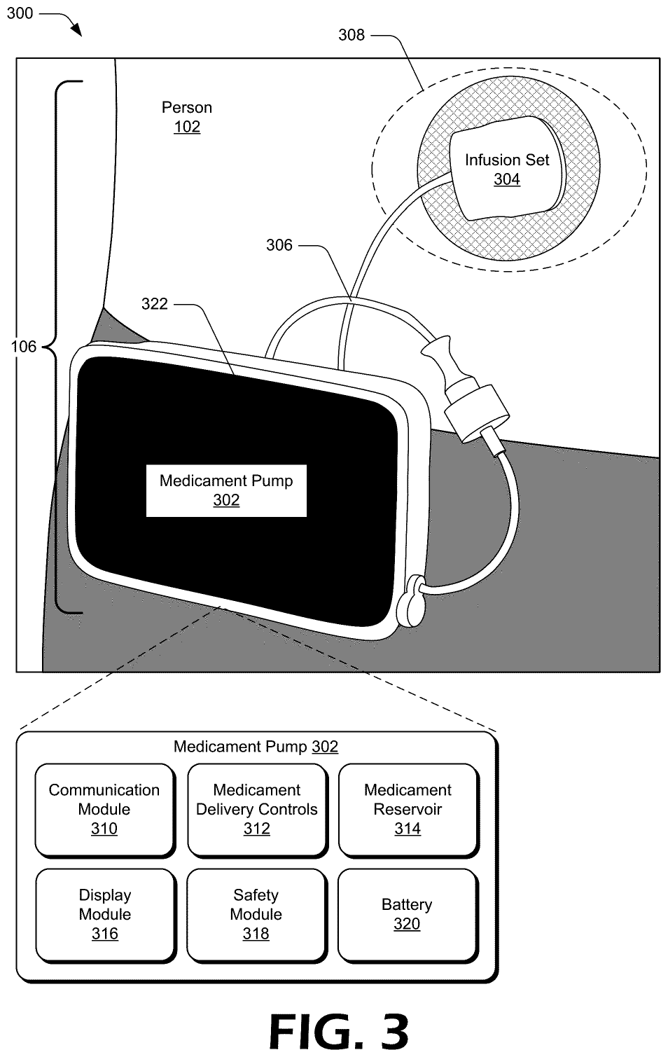

FIG. 3 depicts an example implementation 300 of the medicament delivery system 106 of FIG. 1 in greater detail.

In the example implementation 300, the medicament delivery system 106 includes a medicament pump 302 and infusion set 304. Although the infusion set 304 is depicted with tubing 306 connected to the medicament pump 302, in one or more implementations the infusion set 304 may be tubeless. The medicament delivery system 106 may be configured as a pen, for instance. Broadly speaking, the infusion set 304 is an apparatus configured to subcutaneously deliver medicament—pumped to the infusion set 304 by the medicament pump 302—into the person 102 for absorption by the person 102's bloodstream. In this way, the delivered medicament may be used by the person 102's body to maintain balanced analyte levels, e.g., within a target range of analyte measurements. In one or more implementations, the infusion set 304 includes a cannula that is inserted subcutaneously into skin, such as at infusion site 308 of the person 102 in the example implementation 300. Accordingly, the infusion set 304 may administer doses of medicament through the skin of the person 102, e.g., in a continuous manner and at programmable rates. As discussed herein, for instance, basal and/or bolus doses of insulin may be administered through the skin of the person 102 via the infusion set 304. Alternatively or in addition, the medicament delivery system 106 may administer medicament to the person 102 based on user input, e.g., input received via a user interface of one or more doses of medicament to deliver and/or input received via a computing device 108.

As illustrated, the infusion set 304 includes an adhesive pad which affixes the apparatus to the person 102 for a period of time. In one or more implementations, the infusion set 304 is applied to the infusion site 308 using a separate applicator (not shown). In operation, this applicator may inject the infusion set 304's cannula into the person 102's skin at the infusion site 308 and also affix the adhesive pad to the infusion site 308 to secure the infusion set 304 to the person 102 for a period of use. In at least some implementations, for instance, infusion sets may be disposable such that they are designed for removal after a prescribed and/or recommended period of time and for replacement with a new set that is applied to the person 102 and attached to the medicament pump 302. In any case, the medicament pump 302 is configured to deliver medicament doses to the person 102 via infusion sets, such as the illustrated infusion set 304.

In the example implementation 300, the medicament pump 302 includes communication module 310, medicament delivery controls 312, medicament reservoir 314, display module 316, safety module 318, and battery 320. In implementation, the medicament pump 302 may be configured in various ways, such as with some of these components while others are housed or otherwise implemented in separate devices. Alternately or additionally, the medicament pump 302 may include additional or alternate components without departing from the spirit or scope of the techniques described herein.

The communication module 310 is configured to transmit data to and receive data from other devices, such as the computing device 108. In one or more implementations, the communication module 310 establishes communicative couplings with such other devices to enable transmission and receipt of data. By way of example, the communication module 310 may establish, or otherwise facilitate establishing, links or channels of communication with those other devices. The links or channels may be configured in various ways, including but not limited to Bluetooth (e.g., Bluetooth Low Energy links), Near Field Communication (NFC), 5G or other cellular, and Wi-Fi, to name just a few. Such communicative couplings enable the medicament pump 302 to communicate over different networks, such as the network 116 and/or securely within a closed loop system which includes, for example, the analyte monitoring device 104 and at least one computing device 108.

Once a communicative coupling is established, the communication module 310 can cause data to be transmitted over the established coupling and/or can receive data from other devices over the established coupling. Additionally or alternately, the communication module 310 may be configured to establish connections over wired communication channels—such as via a USB cord connected to the medicament pump 302 and another device—and also configured to transmit and/or receive data over such a wired coupling. The communication module 310 may be configured in various ways to enable the medicament pump 302 to communicate with other devices.

As one example, the communication module 310 enables the medicament pump 302 to receive instructions 120 and/or other instructions from the computing device 108 and/or the glycemic control system 110 for controlling delivery of medicament to the person 102. For instance, the communication module 310 enables the medicament pump 302 to receive instructions that instruct the medicament pump 302 regarding delivery of a basal rate of insulin to the person 102, updates to the basal rate of insulin, delivery of bolus doses of insulin to the person 102 (e.g., an amount to bolus within a limited time), and so forth. The computing device 108 may send a variety of communications to the medicament delivery system 106 for controlling medicament delivery without departing from the spirit or scope of the techniques described herein.

The medicament delivery controls 312 represent any hardware, software, and/or mechanical components of the medicament pump 302 that cause medicament to be pumped (or otherwise extracted) from the medicament reservoir 314 so that it flows through the infusion set 304 and into the person 102. Moreover, the medicament delivery controls 312 are further configured to cause the medicament to be pumped or otherwise extracted from the medicament reservoir 314 in accordance with medicament dose instructions, e.g., the instructions 120 from the glycemic control system 110 which specify one or more deliveries of the medicament. The medicament reservoir 314 is configured to house an amount of medicament, which by leveraging the functionality of the medicament pump 302 may be subcutaneously delivered via the infusion set 304. The medicament reservoir 314 may be replaceable or otherwise configured so that the amount of medicament can be replenished in the medicament reservoir 314. The medicament reservoir 314 may be configured in various ways (e.g., different shapes, different materials, differently removable, and so on) without departing from the spirit or scope of the described techniques.

The display module 316 is configured to cause display of information via a display device 322 of the medicament pump 302. The display module 316 may generate one or more user interfaces for display via the display device 322. By way of example, the display module 316 may cause display via the display device of a user interface for setting up a wireless connection with the computing device 108. Additionally or alternately, the display module 316 may cause the display device 322 to display analyte measurements (e.g., in a similar manner as they are displayed via a health monitoring application of the computing device 108), trend arrows (e.g., regarding an identified trend in the analyte measurements), alerts (e.g., about the analyte measurements, other physiological conditions, operability of the medicament delivery system 106, operability of components of the glycemic control system 110, status of a wireless connection with the computing device 108, and so on), pump set up interfaces, indications of being out of communication range from different devices (e.g., the computing device 108 or the analyte monitoring device 104), and so forth. The display module 316 may thus cause a variety of information to be displayed via the display device 322 of the medicament pump 302.

Safety module 318 is configured to provide one or more safeguards to control delivery of medicament so that the delivery is not harmful; in other words, so that the medicament is delivered safely. By way of example, the safety module 318 may contain or otherwise enforce delivery limits, such as maximum and minimum rates of delivery over different periods of time. These limits are effective to prevent erroneous delivery instructions from the glycemic control system 110 from harming the person 102, such as when an error transmitting the instructions affects their contents or when an error in a prediction made by one or more machine learning models dangerously affects those instructions. For instance, the safety module 318 may limit an amount or rate of medicament that the medicament pump 302 delivers to a threshold amount or threshold rate, even if instructions received from the glycemic control system 110 instruct the medicament pump 302 to deliver more than the threshold amount. Similarly, the safety module 318 may also prevent the medicament pump 302 from delivering less than a threshold amount of medicament or delivering medicament at less than a threshold rate, even if instructions received from the computing device 108 instruct the medicament pump 302 to deliver less than the threshold amount.

In addition, the safety module 318 is configured to continue operating the medicament pump 302 in the absence of instructions from the glycemic control system 110 or the computing device 108, e.g., in the absence of instructions describing how much medicament to deliver and when. The safety module 318 may be configured to continue operating the medicament pump 302 to deliver medicament to the person 102 when the glycemic control system 110 is out of communication range, for example. The safety module 318 may have access to logic, default settings, or settings entered as part of a set up process, to name just a few, that control how much medicament the medicament pump 302 is to deliver when instructions are not being received from the glycemic control system 110. The safety module 318 may perform various additional or different safeguards to ensure that an amount of medicament delivered is not harmful to the person 102.

The battery 320 is configured to provide power for operating the medicament pump 302, such as to power the communication module 310 to send and receive data, to power the medicament delivery controls 312 to cause delivery of medicament from the medicament reservoir 314 to the person 102 via the infusion set 304, to power the display module 316 to display information via the display device 322, and so forth. The battery 320 may be rechargeable (e.g., via a USB charging port or wirelessly) or replaceable. It is to be appreciated that the battery 320 may be configured in various ways.

Although not depicted, the medicament delivery system 106 or another device (e.g., the analyte monitoring device 104) may be configured with a medicament sensor (e.g., an insulin sensor). Such a medicament sensor may be applied to skin or inserted subcutaneously to measure a systemic level of the medicament, e.g., in the person 102. Accordingly, the medicament sensor may be included as part of the infusion set 304, the analyte monitoring device 104, or may be separately applied. In any case, such a sensor may be used in connection with the safety module 318 and/or with dose prediction functionality of the glycemic control system 110. In this way, medicament measurements produced using the medicament sensor can be used to prevent or detect an overdose in the medicament, e.g., to prevent a person who is receiving insulin from experiencing an episode of hypoglycemia. By way of example, the safety module 318 may shut off medicament delivery by the medicament pump 302 based on the medicament measurements, e.g., if the safety module 318 detects that the level of medicament surpasses a predetermined threshold. Based on the medicament measurements, the glycemic control system 110 may also or alternately send instructions to the medicament delivery system 106, instructing it to cease medicament delivery. In one or more implementations, the safety module 318 and/or the computing device 108 may also trigger alerts if the level of medicament surpasses the predetermined threshold. Physiologically, different thresholds may be determined for different persons based on their medicament sensitivities (e.g., insulin sensitivities), such that the above-discussed shut down, alerts, or cease to medicament delivery may be triggered at different medicament levels for the different persons.

Having considered an example of an environment and example devices, consider now a discussion of some examples of details of the techniques for location-aided glycemic control in accordance with one or more implementations.

Location-Aided Glycemic Control

FIG. 4 depicts an example of a system 400 that receives and processes sensor data from various sources to generate recommendations for glycemic control based on a location of a user according to steps of one or more algorithms and thus implementing a special-purpose machine. The illustrated system 400 includes from FIG. 1 the glycemic control system 110 having the location prediction engine 122 and the recommendation engine 124.

In this example, the glycemic control system 110 is depicted obtaining the sensor data 118. In accordance with the described techniques, the glycemic control system 110 receives the sensor data 118 from various sources 402, examples of which include global positioning system (GPS) data, Wi-Fi information (e.g., service set identifiers (SSIDs)), Bluetooth Low Energy (BLE) information, data produced using a cellular antenna (e.g., long term evolution (LTE)), microphone data (e.g., sound data), accelerometer data, gyroscope data, magnetometer data, barometer data, ambient or internal temperature data (e.g., produced using temperature sensors), light data (e.g., captured using a device's camera), heart rate data (e.g., produced by a smartphone and/or a smart watch), proximity data (e.g., between devices), humidity data, analyte data (one or more different analytes or different data about a same analyte from different sources), and medicament data, to name just a few. Additional examples of the sensor data 118 from the various sources 402 includes, but is not limited to, measurements of various detected signals (e.g., biopotential measurements such as electrocardiogram (ECG), electromyography (EMG), or electroencephalogram (EEG); acceleration experienced by the person at a location where the analyte augmentation wearable is worn; and optical signals such as photoplethysmogram (PPG) that detect changes in blood volume), measurements of various physiological conditions (e.g., perspiration, temperature, heart rate, oxygen saturation (SpO₂)), or indications of detected events (e.g., exceeding or falling below a threshold, detecting the presence or absence of a particular compound), to name just a few. As noted above, the analyte monitoring device 104, the medicament delivery system 106, the computing device 108, and the IoT 114 may be the sources 402 of such data. Alternatively or in addition, the glycemic control system 110 receives the sensor data 118 from other sources 402 which may include or be associated with one or more various types of sensors.

In the illustrated example, the sensor data 118 includes first sensor data 404 and second sensor data 406. In one or more implementations, the location prediction engine 122 predicts a location 408 of a user based on at least two types of sensor data, e.g., the first sensor data 404 and the second sensor data 406. For instance, the location prediction engine 122 predicts the location 408 of the user based on both GPS data and Wi-Fi information (e.g., an SSID of a network accessible to the computing device 108), based on both Wi-Fi information and sound data, based on both Wi-Fi information and light data, based on both Wi-Fi information and heart rate data, to name just a few. It is to be appreciated that the location prediction engine 122 can be configured to receive as input, and process, various combinations of at least two types of the data described above and below to predict the location 408 of a user. Certainly, the location prediction engine 122 can use more than two types of data to predict the location 408 of the user in one or more implementations, e.g., more than the first sensor data 404 and the second sensor data 406.

In at least one implementation, the location prediction engine 122 predicts the location 408 of a user (e.g., the person 102) based on a location of a computing device 108 associated with the user. For example, the location prediction engine 122 predicts the location 408 of the user based on the location of a mobile phone or a smart watch of the user. In such implementations, this is based on the assumption that the user is proximate the associated computing device, e.g., the computing device is worn by the user or carried in the user's clothing or an accessory. In other words, the location prediction engine 122 predicts (or detects) the location of the user's computing device 108 (e.g., the user's mobile phone or smart watch) then ascribes the device's location to the user. Alternatively or in addition, the location prediction engine 122 predicts (or detects) the location of the user's computing device 108 (e.g., the user's mobile phone or smart watch) then uses sensor data 118 (e.g., proximity data, infrared data, temperature data, etc.) obtained from the computing device 108 to further refine the location and/or determine a location of the user relative to the computing device 108. This may be the case when the computing device 108 is not worn by the user or is not “on” the user, such as when the user is sleeping or showering. In one or more implementations, the location prediction engine 122 generates a “coarse” prediction of the location 408 of the user using the first sensor data 404, and the location prediction engine 122 generates a “refined” prediction of the location 408 using the second sensor data 406 (and based on the coarse location prediction).

In the illustrated example, the location prediction engine 122 includes location predictor 410 and location confirmation engine 412. The location confirmation engine 412 is illustrated with dashes, indicating that its logic is optionally used in one or more implementations. In accordance with the described techniques, the location predictor 410 corresponds to the logic the location prediction engine 122 uses to predict the location 408 of a user based on the sensor data 118, according to one or more algorithms and thus implementing a special-purpose machine. The location predictor 410 is configured in a variety of ways without departing from the spirit or scope of the described techniques. For example, the location predictor 410 may be configured as or include one or more machine learning models. Alternatively or in addition, the location predictor 410 includes or has access to a mapping of locations (e.g., restaurants, gyms, houses, stores, roads, and other businesses) to geographic coordinates, such that given precise geographic coordinates the location predictor 410 can ascribe the user's presence to a location according to the mapping.

In at least one implementation, the first sensor data 404 may not be suitable to enable the location predictor 410 to predict the user's geographic coordinates precisely enough to discriminate between at least two proximate locations. Instead, the first sensor data 404 may be suitable to predict an area (e.g., a radius around a point) where the user is likely to be located, such that the area includes at least two candidate locations. Thus, in one or more implementations, the location prediction engine 122 (e.g., the location predictor 410) uses at least the second sensor data 406 to discriminate between the candidate locations and select the location 408 from the multiple candidate locations. In a scenario where a restaurant and a gym neighbor one another, for instance, the location predictor 410 uses the second sensor data 406 to narrow down the candidate locations to select either the restaurant or the gym. This is notable because the activities that users participate in at different locations can have drastically different effects on a user's analyte levels, e.g., eating a pizza can have a drastically different effect on a person's glucose than exercise.

The location confirmation engine 412 is configured to confirm a location predicted by the location predictor 410. In one or more implementations, the location predictor 410 predicts the location 408 based on the first sensor data 404 and the location confirmation engine 412 attempts to confirm the prediction of the location 408 based on the second sensor data 406. Based on the second sensor data 406, for instance, the location confirmation engine 412 either confirms the prediction of the location 408 or rejects the prediction. In scenarios where the prediction is rejected, the location prediction engine 122 may prompt a user to select a location, such as to select from a plurality of candidate locations or to confirm the predicted location. User involvement may be requested in one or more other scenarios as well, such as a first time that a particular location is predicted for a user. Alternatively or in addition, the location predictor 410 predicts the location 408 based on both the first sensor data 404 and the second sensor data 406, and the location confirmation engine 412 attempts to confirm the prediction of the location 408 based on both the first sensor data 404 and the second sensor data 406. Alternatively or in addition, the location predictor 410 predicts the location 408 based on both the first sensor data 404 and the second sensor data 406, and the location confirmation engine 412 attempts to confirm the prediction of the location 408 based third sensor data (not shown). Alternatively or in addition, the location predictor 410 and the location confirmation engine 412 may predict the location and attempt to confirm the predicted location, respectively, based on different combinations of data without departing from the spirit or scope of the described techniques. Further, it is to be appreciated that the location prediction engine 122 may include or otherwise have access to various types of logic to predict the location 408 of a user (e.g., the person 102) based on the sensor data 118 in accordance with the described techniques. In this example, the location prediction engine 122 outputs the location 408.

Given the location 408, the system 400 predicts an activity 414 of the user, such that a recommendation for glycemic control can be generated based on the predicted activity 414. Here, the recommendation engine 124 is depicted obtaining the location 408. In the illustrated example, the recommendation engine 124 includes activity predictor 416 and activity-to-recommendation module 418. It is to be appreciated that the recommendation engine 124 may include more, different, or fewer modules without departing from the spirit or scope of the described techniques.

The activity predictor 416 is depicted outputting the activity 414. In accordance with the described techniques, the activity predictor 416 determines and outputs the activity 414 the user is participating in or likely to be participating in based on the location 408, according to one or more algorithms and thus implementing a special-purpose machine. In one or more variations, the activity predictor 416 predicts the activity 414 based on the predicted location 408 without using other sensor data 118 (e.g., analyte data) or user interaction to confirm the activity. In at least one other variation, the activity predictor 416 predicts the activity 414 based on the predicted location 408 and at least one other type of data, e.g., the sensor data 118 and/or based on data describing user interaction with a device. Examples of such sensor data 118 include sound data (e.g., indicative of sounds confirming that a user is sleeping), light data (e.g., indicative of a dark location which is associated with a higher likelihood that the user is sleeping), accelerometer data (e.g., indicative of a positioning of a device on a nightstand which is associated with a higher likelihood that a user is sleeping), and so on. Examples of such user interaction include user interaction to confirm a predicted activity 414, to input the activity, and so forth. As noted above and below, different activities that persons participate in can have vastly different effects on glucose. In other words, a person's glycemic response is different to different activities that they participate in. Consequently, the recommendation engine 124 recommends different mitigating therapies based on the activity 414.

Examples of activities include, but are not limited to, eating, sleeping, exercising (e.g., aerobic, anerobic, partially aerobic and partially anerobic, high-intensity interval training, running, rowing, hiking, biking, weightlifting, yoga, Pilates, sports, and so forth), working (e.g., potentially causing stress), showering, watching a movie, watching television, attending an event (e.g., a sporting event or a concert), dancing, reading, cleaning, taking care of children, or driving, to name just a few.

In the illustrated example, the activity predictor 416 is also depicted outputting activity state 420. The activity state 420 is illustrated with dashes to indicate it is optionally predicted by the activity predictor 416 in one or more implementations. The activity state 420 is useful because a therapy recommended at different times relative to an activity (e.g., before, during, and after) may be different. For example, a therapy recommended prior to eating a meal may be different from a therapy recommended while eating a meal or after eating a meal. Similarly, a therapy recommended prior to exercising may be different from a therapy recommended while exercising or after exercising. Thus, in one or more implementations, the activity predictor 416 predicts a pre-activity state, a during activity state, or a post-activity state along with predicting the activity 414.

By way of example, the location 408 of a user may be predicted as in a car (e.g., driving), and based on additional user data, the activity predictor 416 may predict that the user is on the way to the gym. Examples of such additional data may include, but are not limited to, data describing historical behaviors of the user, calendar data, streaming (e.g. music) data, data from a mapping application (e.g., outputting or determining a route to the gym, detecting that the user is driving on a route that can be taken to the gym, etc.), data from an application associated with the location (e.g., reservation or scheduled appointment on the application), and so on. Accordingly, in this example, the activity predictor 416 can predict that the activity 414 is exercising, and that the activity state 420 is ‘pre-activity’ or pre-exercising. As additional data is received, the activity predictor 416 may update the activity state 420 or the activity 414. In the continuing example, for instance, the activity predictor 416 may update the activity state 420 to a ‘during activity state’, or exercising, when additional data is received and processed, such as data describing that the user has parked a car at the gym, the user has checked into the gym, the user has selected to start a workout on a smartwatch, the computing device of the user has connected to another device (e.g., established a Wi-Fi connection with a wireless network of the gym, established a Bluetooth connection with a chest strap heart monitor), or the user has selected to initiate playback of a workout playlist, to name just a few.

Based on the activity 414, and in at least one implementation the activity state 420, the activity-to-recommendation module 418 generates a recommendation 422. In one or more implementations, the activity-to-recommendation module 418 predicts a glycemic response 424 of the user to the predicted activity 414, determines that the glycemic response 424 of the user to the predicted activity will cause an adverse health event, and determines a mitigating therapy 426 to prevent or mitigate the adverse health event corresponding to the glycemic response 424. Adverse health events, for example, may include hypoglycemic events, hyperglycemic events, and any other potentially harmful events associated with the user's glucose levels. The activity-to-recommendation module 418 then generates the recommendation 422 to provide the mitigating therapy 426 and/or notification of the mitigating therapy 426 to the user (e.g., the person 102). The activity-to-recommendation module 418 may be configured in a variety of ways without departing from the spirit or scope of the described techniques.

It is to be appreciated, therefore, that in some configurations the glycemic control system 110 continuously detects the location 408 of the user, and predicts activities 414 associated with the location. However, not all activities cause a glycemic response 424 which may result in an adverse health event. Thus, in one or more implementations, the recommendation 422 to control the glycemic response 424 is only generated in instances where the glycemic control system 110 determines that the activity will cause a glycemic response 424 which will cause an adverse health event. As an example, the glycemic control system 110 may determine that the user is at her office, and predict that the user will be performing an activity associated with the user's office, such as writing an email. In this case, however, the glycemic control system 110 can determine that this activity will not cause a glycemic response 424 that results in an adverse health event. As such, the glycemic control system may not generate a recommendation 422 in this instance even though an activity is predicted.

In one or more implementations, for example, the activity-to-recommendation module 418 is configured as a machine learning model that receives as input the activity 414 (and the activity state 420 in some implementations) and outputs the recommendation 422. In such implementations, the activity-to-recommendation module 418 may be trained using training data that pairs activities (and activity state in some implementations) with clinically recommended therapies. By way of example, the machine learning model may be exposed to an activity (e.g., receive it as input) and attempt to output a recommendation for a mitigating therapy. The system may then compare the output to the recommended therapy paired with the input in the training data. Based on this comparison, internal weights of the machine learning model may be adjusted. For instance, if the output matches the therapy paired in the training data, then weights may be adjusted (e.g., strengthened) to encourage the output during future iterations. However, if the output does not match the therapy paired in the training data, or is identified as being detrimental to the user's health given the activity, then the weights may be adjusted to discourage the output during future iterations. The machine learning model may be thusly trained over numerous iterations.

Alternatively or in addition, the activity-to-recommendation module 418 may obtain the glycemic response 424 and the mitigating therapy 426, such as from a mapping of activities (and states in some implementations) to glycemic responses 424 to those activities and the mitigating therapies 426. By way of example, a library may include a mapping that maps different activities to a respective glycemic response 424 and one or more respective mitigating therapies. This information may be stored in a database, for example. In one or more implementations, the mapping may be approved by one or more clinicians. For instance one or more clinicians may approve or otherwise provide a recommended mitigating therapy 426 for an activity 414, because the activity is likely to cause a particular glycemic response 424 and the recommended mitigating therapy 426 is clinically recommended for mitigating the glycemic response.

This information may be input to the system (e.g., into a database) and maintained. In one or more implementations, such a mapping may be maintained in computer-readable storage media of a computing device 108 of the user (e.g., the user's mobile phone). Alternatively or in addition, the mapping may be maintained remote from the computing device 108, such as in a database associated with the glycemic control system 110 and/or the health monitoring platform 112. Either or both of remote and local mappings may be updated as new therapies and activities are added and/or the therapies that are mapped to various activities are updated, e.g., when clinicians recommend a different therapy for an activity than was previously clinically recommended. In one or more implementations, the mapping may be personalized to a user, e.g., to map activities to therapies that have worked for the user previously to control the user's glycemic response to the activity. In such implementations, the mapping may be provided by the user (e.g., via user input to the user's computing device), a health care provider (e.g., via a health care provider portal), or determined by the system based on the sensor data 118 (e.g., analyte data describing that an analyte level of the user remained within a range and/or no harmful excursions occurred).

Accordingly, the recommendation 422 is configured to provide the user or notify the user of a mitigating therapy 426 to mitigate the glycemic response 424 to the activity 414 predicted based on the location 408 of the user. The recommendation 422 may include one or more of a variety of therapies to control the user's glycemic response 424 to the predicted activity 414. By way of example, and not limitation, the recommendation 422 may include dosage and/or timing of one or more medicaments, activities to perform (e.g., exercise, contacting a health care professional, discontinuing an activity, resting) and/or timing of those activities, amount and/or timing of one or more foods to consume (e.g., drink a number of ounces of juice or eat a tablet), and so on. The recommendation may recommend a variety of therapies without departing from the spirit or scope of the described techniques.

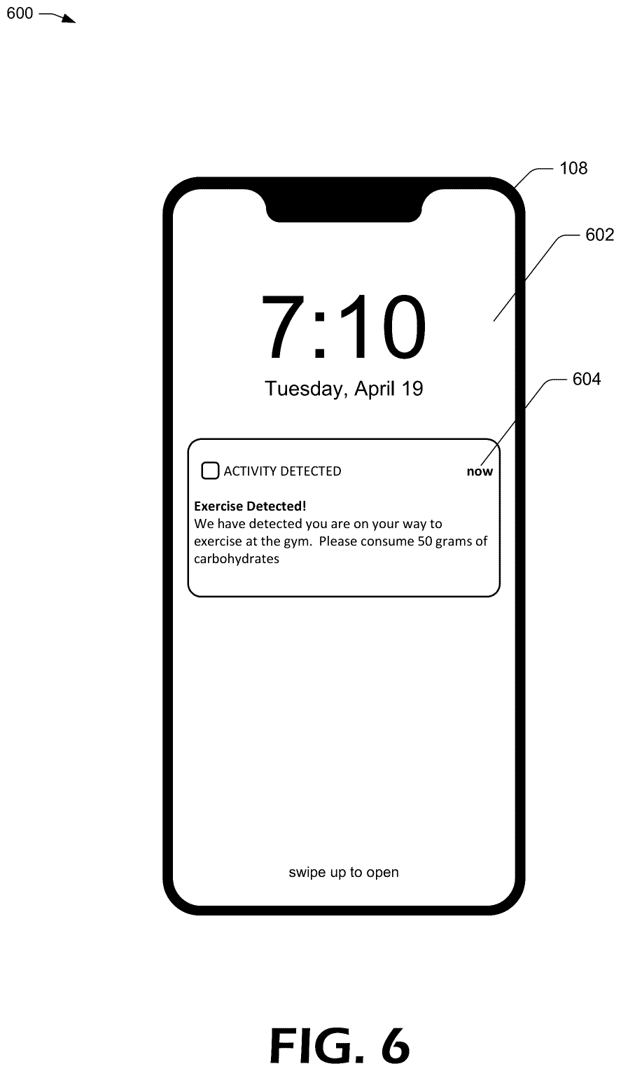

Based on the recommendation 422, controller 428 outputs the instructions 120. For example, the controller 428 provides the instructions 120 to the medicament delivery system 106, the computing device 108, and/or to another device (e.g., an additional medicament delivery system). In one or more implementations, the controller 428 provides the instructions 120 to notify the user about the recommended mitigating therapy 426. For instance, the controller 428 provides the instructions 120 to the computing device 108 to cause the computing device 108 to display or otherwise output the recommendation 422 (or information indicative of the recommendation 422). Examples of notifications that can be output by a computing device 108 are discussed in more detail in relation to FIGS. 6-9.

Alternatively or additionally, the controller 428 provides the instructions 120 to control a device (e.g., the medicament delivery system 106) to provide the recommended mitigating therapy 426. For instance, the controller 428 provides the instructions 120 to the medicament delivery system 106, and the instructions cause the medicament delivery system 106 to administer an amount of medicament to the person 102.

In one or more implementations, the instructions 120 cause the medicament delivery system 106 to administer the medicament (e.g., a dose) to the person 102 automatically, without user input. In such implementations, the devices are operatively connected in a closed loop. In other implementations, the instructions 120 instruct the medicament delivery system 106 to administer the medicament (e.g., a dose) to the person 102, but the system prevents the medicament from being delivered until a validation input is received from the user, e.g., an input indicating that the user allows the medicament to be delivered. Alternatively or additionally, the instructions 120 instruct the medicament delivery system 106 to administer a dose of the medicament, and the medicament delivery system 106 requires user interaction to transfer the dose into the body of the person 102, e.g., a user is required to position the medicament delivery system 106 against the person 102's body and actuate the system (e.g., press a button) to administer the medicament.

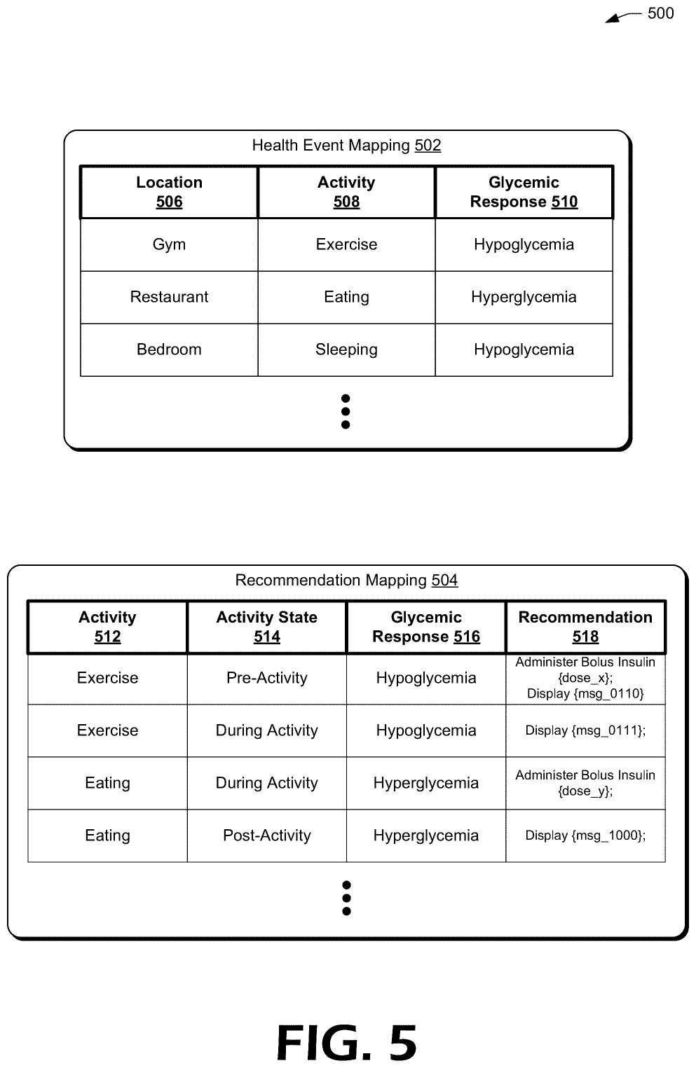

FIG. 5 depicts an example 500 of mappings for location-aided glycemic control. In particular, the illustrated example 500 includes a health event mapping 502 and a recommendation mapping 504. In or more implementations, the health event mapping 502 and the recommendation mapping 504 are stored in storage that is accessible to the glycemic control system 110 (or components thereof), such as in computer readable storage media of the computing device 108, the glycemic control system 110, and or the health monitoring platform 112. For example, the health event mapping 502 and/or the recommendation mapping 504 may be configured as a file and/or a database in one or more implementations. In the illustrated example, the health event mapping 502 maps locations to activities and glycemic responses, and the recommendation mapping 504 maps activities to recommendations. It is to be appreciated that mappings used in connection with the described techniques may map different information than depicted without departing from their spirit or scope.

In this example, the health event mapping 502 includes fields for location 506, activity 508, and glycemic response 510. Thus, the health event mapping 502 includes entries, each of which specifies a location 506 and, for the location 506 of the entry, an activity 508 and a glycemic response 510 to the activity 508. It is to be appreciated that a location in the mapping may be associated with multiple entries, e.g., because multiple different activities may be performed at the location. One example of such a location is a home, where a user may sleep, eat, and exercise. Accordingly, the location may be more specific, e.g., kitchen, bedroom, or garage. By way of example, the activity predictor 416 may leverage the health event mapping 502 to determine the activity 414 based on the predicted location 408.

Here, the recommendation mapping 504 includes fields for activity 512, activity state 514, glycemic response 516, and recommendation 518. Thus, the recommendation mapping 504 includes entries, each of which specifies an activity 512 and an activity state 514. For the activity state 514 of the activity 512, the mapping includes a likely glycemic response 516 and a recommendation 518 to control the glycemic response, e.g., mitigate any harmful effects of the glycemic response. In one or more implementations, the activity-to-recommendation module 418 may leverage the recommendation mapping 504 to generate the recommendation 422 based on the activity 414 and the activity state 420. In the context of notifications that output information corresponding to recommendations 422, consider the following examples.

FIG. 6 depicts an example of a user interface displaying an activity notification. The illustrated example 600 includes from FIG. 1 an example of the computing device 108 displaying an example user interface 602 via a display device, e.g., a touchscreen. Here, the user interface 602 includes an activity notification 604 which indicates that an activity 414 been detected by activity predictor 416.

In this example, the activity corresponds to predicted exercise based on sensor data 118 indicating that the user is travelling to the gym to exercise, e.g., the user is driving or walking to the gym. The activity notification 604 also includes a recommendation 422 which instructs the user to consume 50 grams of carbohydrates to mitigate the likelihood of the user experiencing a hypoglycemic event caused by the exercise. For example, exercise lowers the user's glucose levels, and thus consuming 50 grams of carbohydrates prior to exercise may raise the user's glucose levels such that the user's glucose levels will not fall below the hypoglycemic threshold after exercising.

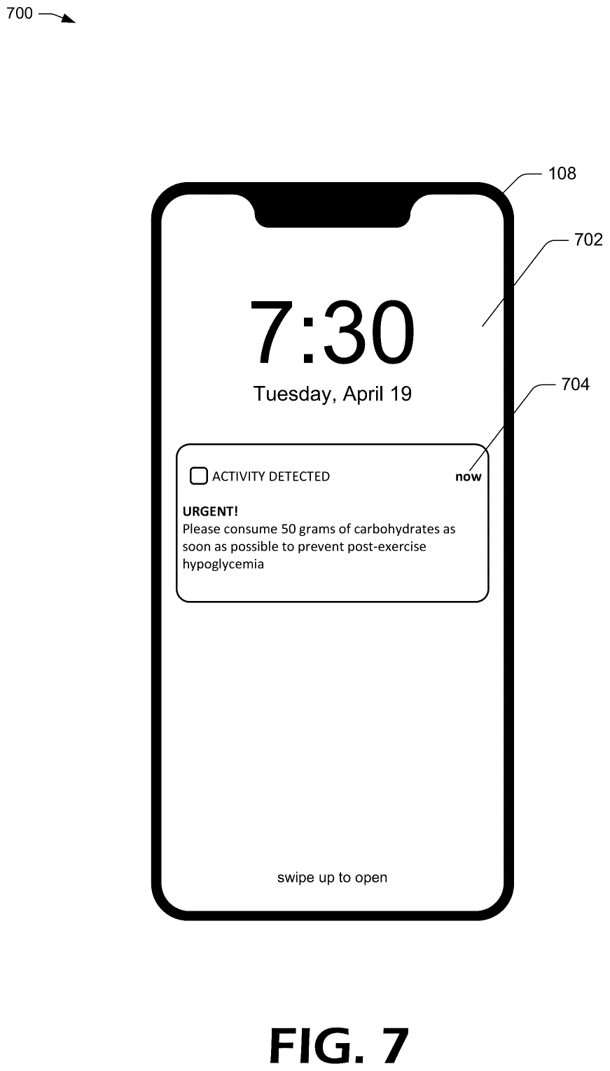

FIG. 7 depicts an additional example of a user interface displaying an activity notification. Like the illustrated example 600, the illustrated example 700 includes from FIG. 1 an example of the computing device 108 displaying an example user interface 702 via a display device, e.g., a touchscreen. Here, the user interface 702 includes an activity notification 704 which indicates that an activity 414 been detected by activity predictor 416. Notification 704 corresponds to a second notification displayed subsequent to the notification 604 of FIG. 6 which again includes a recommendation 422 for the user to consume 50 grams of carbohydrates to mitigate the likelihood of the user experiencing a hypoglycemic event caused by the exercise.

The notification 704 may be displayed based on additional sensor data 118 indicating that the user has now arrived at the gym location or is closer to the gym location than at the time notification 604 was displayed. As compared to notification 604, the notification 704 more aggressively recommends that the user consume 50 grams of carbohydrates to prevent a hypoglycemic event. For example, the notification 704 includes the heading “urgent” and also recommends that the user consume 50 grams of carbohydrates “as soon as possible to prevent post-exercise hypoglycemia”. Notably, the glycemic control system has increased the aggressiveness of the recommendation 422 because the user is now closer to in time to starting the activity.

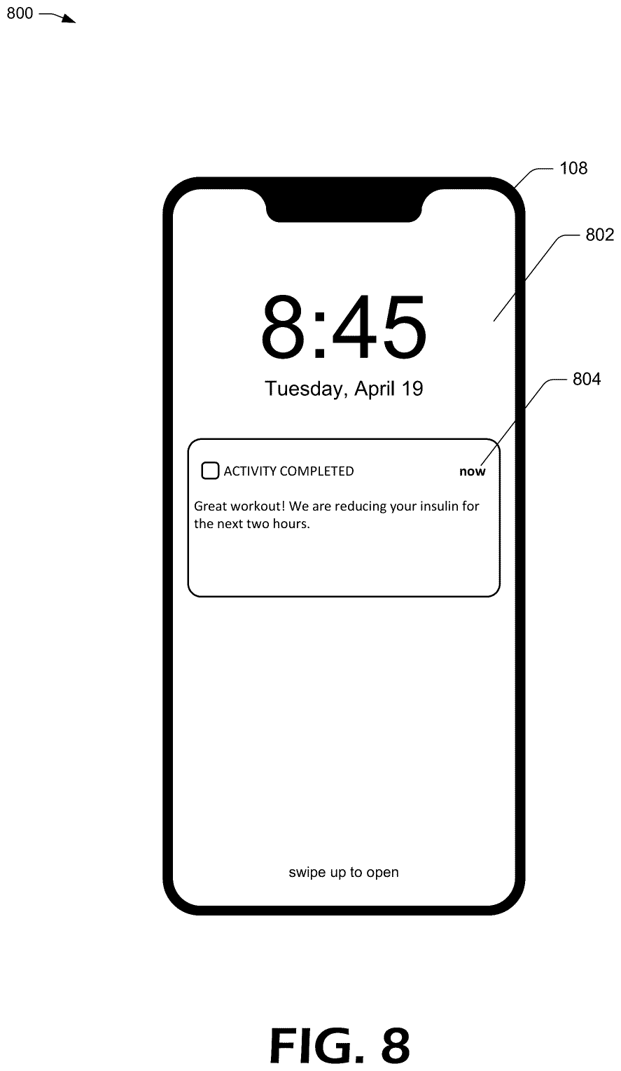

FIG. 8 depicts an additional example of a user interface displaying an activity notification. Like the illustrated examples 600 and 700, the illustrated example 800 includes from FIG. 1 an example of the computing device 108 displaying an example user interface 802 via a display device, e.g., a touchscreen. Here, the user interface 802 includes an activity notification 804 which indicates that an activity 414 been completed. In this case, the activity predictor 416 has detected that the user has completed the exercise activity and is now in the post-exercise state. The activity predictor 416 can detect that the user has completed the activity, for example, based on sensor data 118 indicating that the user is no longer at the gym location. Alternately or additionally, the activity predictor can use other sensor data 118 to determine that the user has completed the exercise, such as heart rate data indicating the user's heart rate has lowered and/or accelerometer data indicating that the user is no longer exercising. In this case, notification 804 indicates that the glycemic control system 110 is reducing the amount of insulin administered to the user for the next two hours. This is done because the exercise may cause the user's glucose levels to decrease, and thus insulin is reduced to prevent the user from experiencing a hypoglycemic event caused by the combination of insulin and exercise.

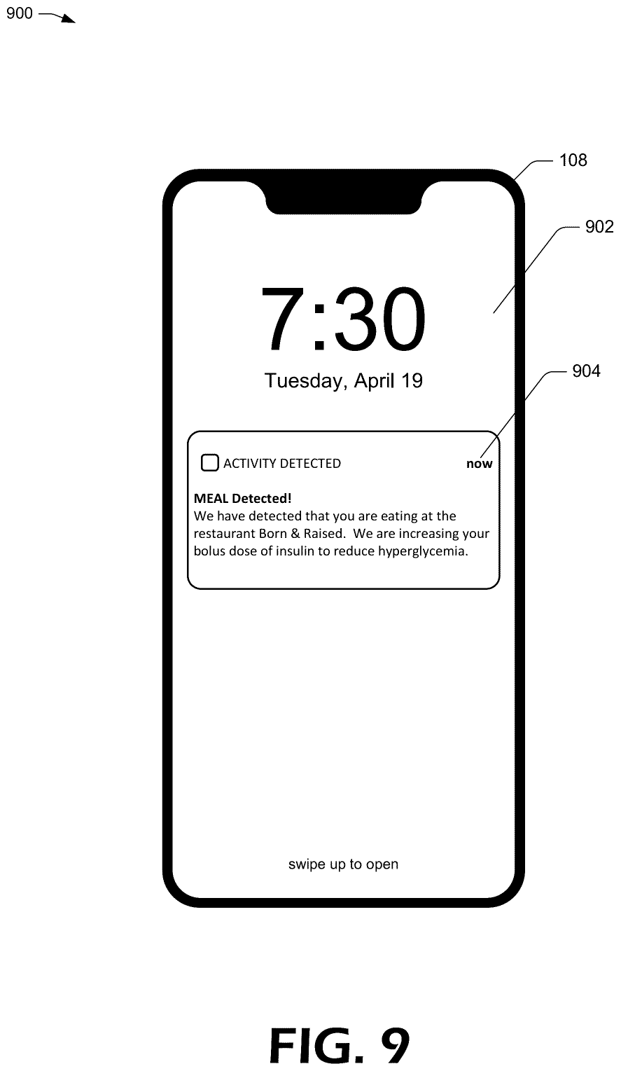

FIG. 9 depicts an additional example 900 of a user interface displaying an activity notification. Like the illustrated examples 600, 700, and 800 the illustrated example 900 includes from FIG. 1 an example of the computing device 108 displaying an example user interface 902 via a display device, e.g., a touchscreen. Here, the user interface 902 includes an activity notification 904 which indicates that an activity 414 been detected.

In this example, the activity corresponds to the user eating at a restaurant and is determined by the activity predictor 416 based on sensor data 118 indicating that the user is at the restaurant location. In this case, notification 904 indicates that the glycemic control system 110 is increasing a bolus dose of insulin administered to the user. This is done because eating at the restaurant may cause the user's glucose levels to increase, and thus insulin is increased to prevent the user from experiencing a hyperglycemic event caused by the combination of insulin and eating a meal at the restaurant.

Notably, the notifications 604, 704, 804, and 904 are depicted as being displayed on a home screen of the user's smartphone. It is to be appreciated, however, that the notifications can be displayed in a variety of different ways and on a variety of different types of devices without departing from the spirt or the scope of the described techniques.

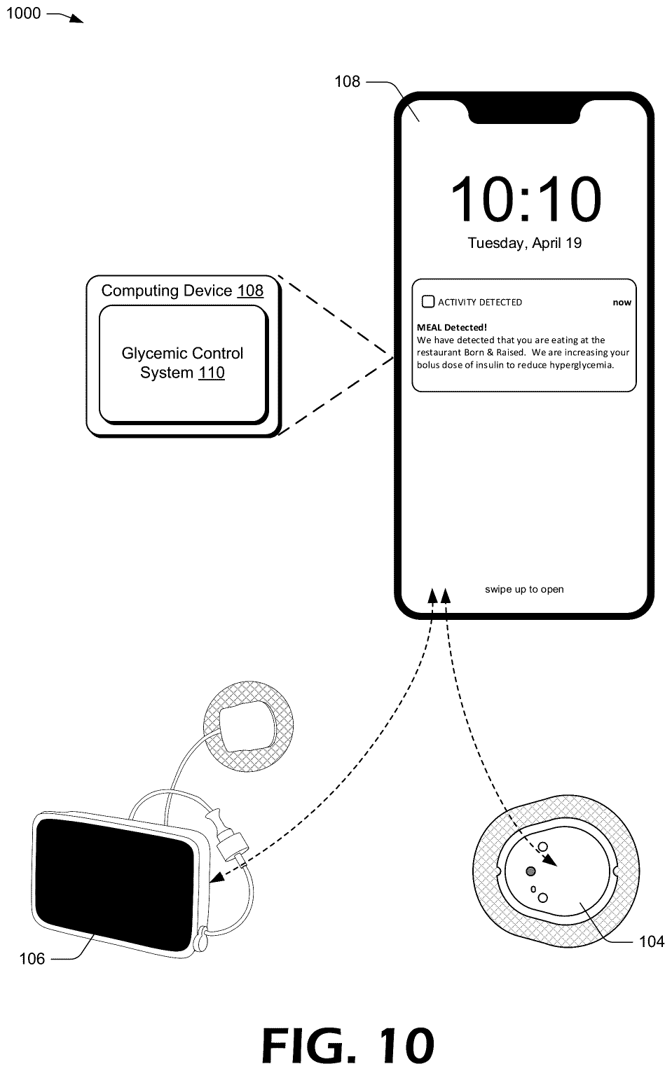

FIG. 10 depicts an example 1000 of a first combination of devices for implementing location-based glycemic control. The illustrated example 1000 includes the analyte monitoring device 104, the medicament delivery system 106, and the computing device 108 having the glycemic control system 110.

In accordance with the described techniques, the analyte monitoring device 104, the medicament delivery system 106, and the computing device 108 may be communicably coupled, e.g., over the network 116 or via some other wireless connection (e.g., BLE), to implement any of the location-based glycemic control techniques described above and below. In variations, the analyte monitoring device 104, the medicament delivery system 106, and the computing device 108 are operably connected to implement location-aided glycemic control as a closed loop system, an open loop system, or a partially open loop system. In this example, the medicament delivery system 106 is depicted as a wearable pump (e.g., insulin pump) having an infusion set that is applied to the person 102 and delivers medicament via the infusion set at an insertion site.

As a closed loop system, the analyte monitoring device 104, the medicament delivery system 106, and the computing device 108 are configured to monitor an analyte of the person 102 wearing the analyte monitoring device 104, recommend therapies based on an activity that the person 102 is predicted to participate in at a predicted location, and administer the recommended therapy (e.g., via the medicament delivery system 106) without user interaction.

By way of contrast, in a partially open system, a user may be required to validate a therapy before it is administered by the system, e.g., the user may be required to provide approval of a recommended medicament dose via a display of the medicament delivery system 106 or the computing device 108 before it is automatically administered by the medicament delivery system 106. Once validated, the medicament delivery system 106 may administer a dose of medicament recommended by the glycemic control system 110. In an open system, the glycemic control system 110 may simply cause a recommended therapy to be output (e.g., displayed) via the computing device 108 (e.g., a display of the computing device 108). In order to administer a recommended dose of the medicament in an open system, the user may provide input to the medicament delivery system 106 specifying the recommended medicament dose and select a control (e.g., a displayed control) to cause the medicament delivery system 106 to administer the dose. By way of contrast to the system in the example 1000, which can be connected to operate in a closed loop configuration that eliminates user interaction, consider the following example in which the medicament delivery system 106 is configured as a pen.

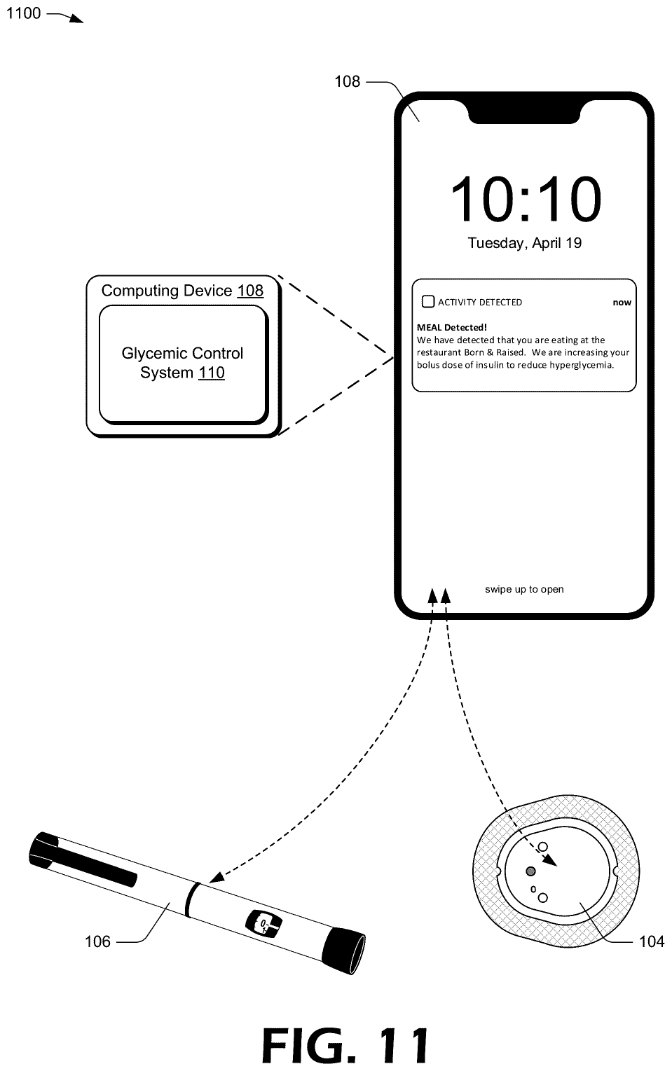

FIG. 11 depicts an example 1100 of a second combination of devices for implementing location-based glycemic control. The illustrated example 1100 includes the analyte monitoring device 104, the medicament delivery system 106, and the computing device 108 having the glycemic control system 110. In contrast to the example 1000, though, in the illustrated example 1100, the medicament delivery system 106 is configured as a pen (e.g., an insulin pen) rather than a wearable pump.

Since the medicament delivery system 106 is configured as a pen, the system in the illustrated example 1100 necessarily involves user interaction. Such interaction may include, for example, interaction to position the pen where medicament stored in the pen can be administered to the person and also interaction to initiate administration of the medicament (e.g., press a button). Thus, when the the analyte monitoring device 104, the medicament delivery system 106, and the computing device 108 are configured as in the example 1100, those devices can be operably connected to implement location-aided glycemic control as an open loop system or a partially open loop system.

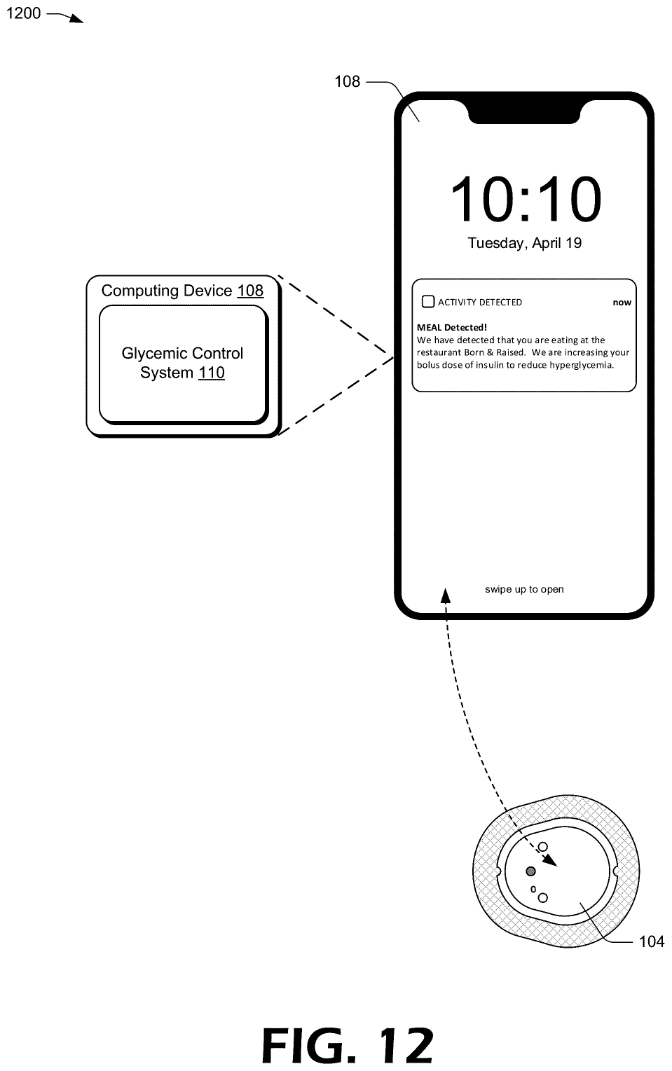

FIG. 12 depicts an example 1200 of a third combination of devices for implementing location-based glycemic control. The illustrated example 1200 includes the analyte monitoring device 104 and the computing device 108 having the glycemic control system 110.

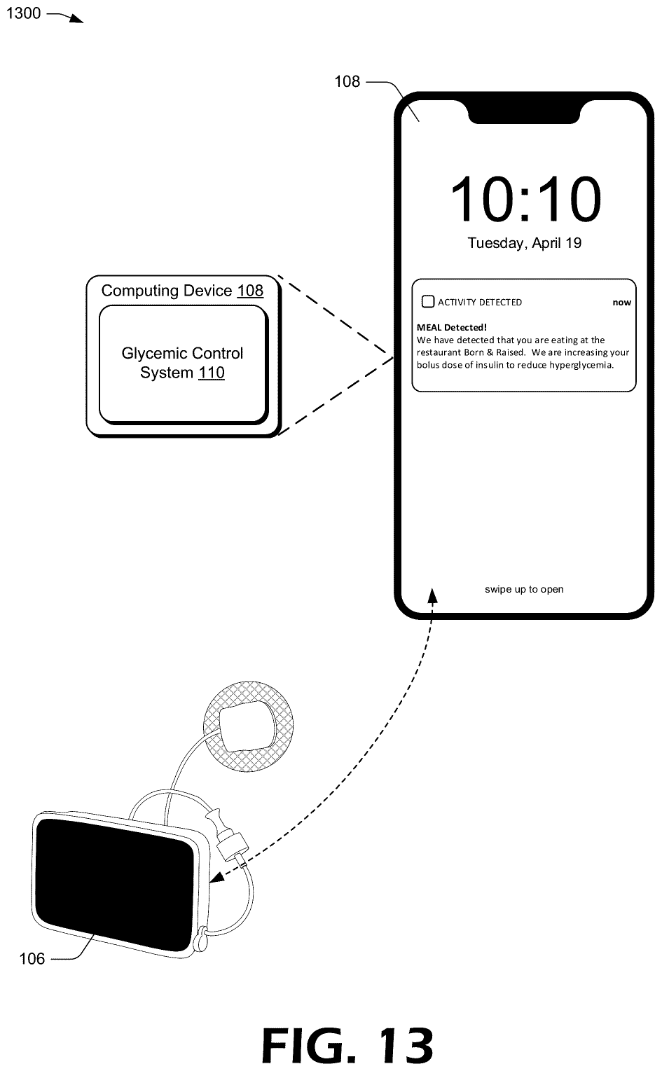

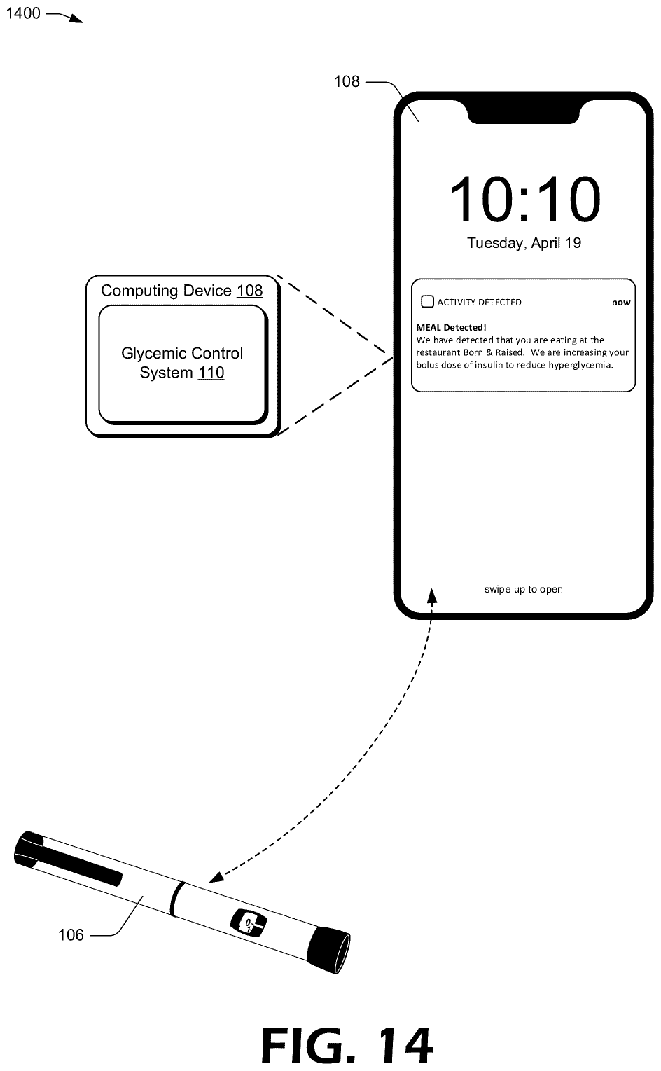

The illustrated example 1200 does not include a medicament delivery system 106, however. This represents a configuration where the analyte monitoring device 104 and computing device 108 are not operably connected to a medicament delivery system 106. In such configurations, the glycemic control system 110 does not communicate instructions 120 to the medicament delivery system 106. Instead, the glycemic control system 110 communicates instructions 120 about recommended therapies to the computing device 108, such as to output (e.g., display) medicament doses and/or other recommended behaviors. In such implementations, a user may interact with a medicament delivery system 106 to specify and/or administer a medicament dose, such as when the medicament delivery system 106 is a syringe that the user fills with a medicament (from a separate container) and injects the medicament into his or her body. In at least one variation of the example 1200 configuration, the sensor data 118 may include analyte data. The glycemic control system 110 can use such analyte data to generate recommendations for mitigating therapies along with using the predicted location and an activity at the location. In contrast to this example, consider the following examples of FIGS. 13 and 14, which do not include the analyte monitoring device 104.

FIG. 13 depicts an example 1300 of a fourth combination of devices for implementing location-based glycemic control. The illustrated example 1200 includes the medicament delivery system 106 and the computing device 108 having the glycemic control system 110.

The illustrated example 1300 does not include an analyte monitoring device 104, however. This represents a scenario where the medicament delivery system 106 and the computing device 108 are not operably connected to the analyte monitoring device 104. In such configurations, the glycemic control system 110 is capable of generating recommendations for therapy without using analyte data. Instead, the glycemic control system 110 predicts a location of a user associated with the computing device 108 and wearing the medicament delivery system 106 (e.g., a pump), and the glycemic control system 110 predicts an activity of the user at the location using other data. Examples of such other data are discussed above, but may include, for instance, GPS data and data produced by other sensors of the computing device. Based on the location and activity predictions generated by the glycemic control system 110 using this other data, the glycemic control system 110 can further generate a recommendation for a mitigating therapy 426.

Like the example illustrated in FIG. 10, the medicament delivery system 106 and the computing device 108 may be operably connected to implement location-aided glycemic control as a closed loop system, an open loop system, or a partially open loop system. As a closed loop system, for instance, the medicament delivery system 106 and the computing device 108 are configured to enable therapies to be recommended based on an activity that the person 102 wearing the medicament delivery system 106 is predicted to participate in at a predicted location, and administer the recommended therapy (e.g., via the medicament delivery system 106) without user interaction.

By way of contrast, in a partially open system, a user may be required to validate a therapy before it is administered by the system, e.g., the user may be required to provide validation of a recommended medicament dose via a display of the medicament delivery system 106 or the computing device 108 before it is automatically administered by the medicament delivery system 106. Once validated, the medicament delivery system 106 may administer a dose of medicament recommended by the glycemic control system 110.

In an open system, the glycemic control system 110 may simply cause a recommended therapy to be output (e.g., displayed) via the computing device 108 (e.g., a display of the computing device 108). In order to administer the recommended dose of the medicament in an open system, the user may provide input to the medicament delivery system 106 specifying the recommended medicament dose and select a control (e.g., a displayed control) to cause the medicament delivery system 106 to administer the dose. By way of contrast to the system in the example 1300, which can be connected to operate in a closed-loop configuration that eliminates user interaction, consider the following example in which the medicament delivery system 106 is configured as a pen.

FIG. 14 depicts an example 1400 of a fifth combination of devices for implementing location-based glycemic control. The illustrated example 1200 includes the medicament delivery system 106 and the computing device 108 having the glycemic control system 110. In contrast to the example 1300, in the example 1400, the medicament delivery system 106 is configured as a pen (e.g., an insulin pen) rather than a wearable pump.

Like the illustrated example 1300, the example 1400 does not include an analyte monitoring device 104, however. This represents a scenario where the medicament delivery system 106 (e.g., pen) and the computing device 108 are not operably connected to an analyte monitoring device 104. In such configurations, the glycemic control system 110 is capable of generating recommendations for therapy without using analyte data. Instead, the glycemic control system 110 predicts a location of a user associated with the computing device 108 and the medicament delivery system 106 (e.g., a pen), and predicts an activity of the user at the location using other data. Examples of such other data are discussed above.

Based on the location and activity predictions generated by the glycemic control system 110 using this other data, the glycemic control system 110 can further generate a recommendation for a mitigating therapy 426. Although the instructions 120 for administering such a therapy may be communicated to the medicament delivery system 106 configured as a pen (e.g., a dose to deliver), at some point user interaction is required in order to administer medicament to the user. As noted above, such user interaction may involve merely positioning the pen on the body of the person where the medicament is to be delivered and selecting a control to administer the medicament. Alternatively, such user interaction may involve validating a recommended dose, and then positioning the pen on the body of the person where the medicament is to be delivered and selecting a control to administer the medicament.

Having discussed exemplary details of the techniques for location-aided glycemic control, consider now some examples of procedures to illustrate additional aspects of the techniques.

Example Procedures

This section describes examples of procedures for location-aided glycemic control. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a glycemic control system, such as glycemic control system 110.

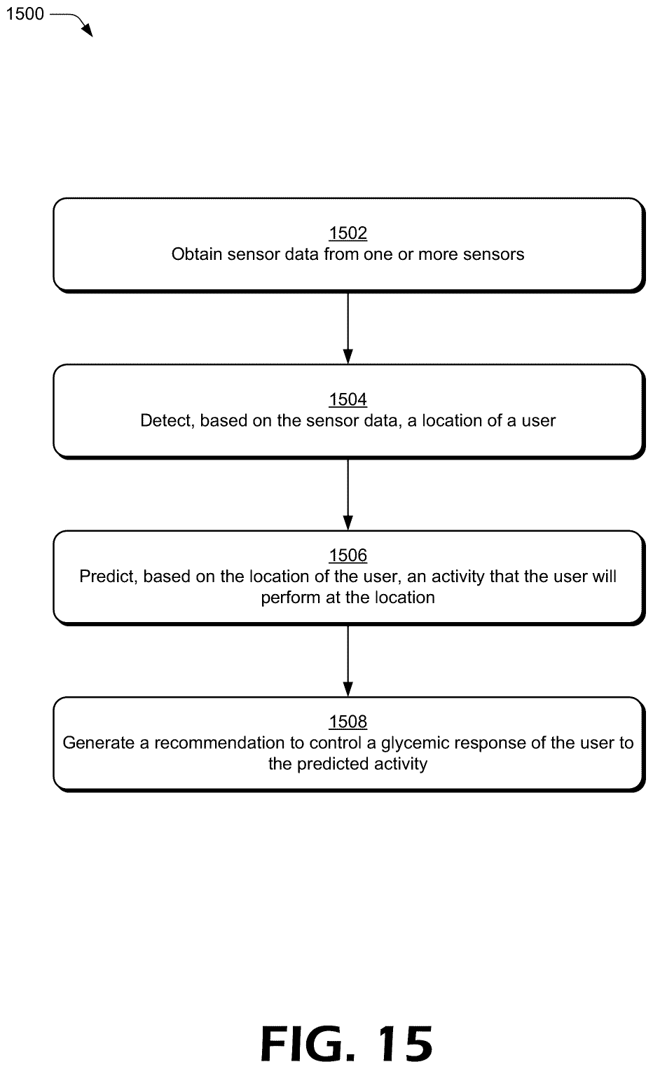

FIG. 15 is a flow diagram depicting an algorithm as a step-by-step procedure 1500 that is performable by a glycemic control system to provide location-aided recommendations for controlling a glycemic response.

Sensor data is obtained from one or more sensors (block 1502). By way of example, the glycemic control system 110 obtains the sensor data 118 from one or more sensors. In accordance with the described techniques, the glycemic control system 110 can receive the sensor data 118 from various sources 402, examples of which include global positioning system (GPS) data, Wi-Fi information (e.g., service set identifiers (SSIDs)), Bluetooth Low Energy (BLE) information, data produced using a cellular antenna (e.g., long term evolution (LTE)), microphone data (e.g., sound data), accelerometer data, gyroscope data, magnetometer data, barometer data, ambient or internal temperature data (e.g., produced using temperature sensors), light data (e.g., captured using a device's camera), heart rate data (e.g., produced by a smartphone and/or a smart watch), proximity data (e.g., between devices), humidity data, analyte data (one or more different analytes or different data about a same analyte from different sources), and medicament data, to name just a few.

A location of the user is detected based on the sensor data (block 1504). By way of example, based on the sensor data 118, the location prediction engine 122 detects a location 408 of a user, e.g., the person 102 wearing the analyte monitoring device 104. In one or more implementations, the location prediction engine 122 detects the location 408 of the user based on at least two different types of the sensor data 118. For instance, the location prediction engine 122 detects the location 408 of the user based on both GPS data and Wi-Fi information (e.g., an SSID to which the computing device 108 of the user is connected) or based on Wi-Fi information and sound data. In one or more implementations, the location prediction engine 122 uses the first sensor data to detect a location of the user and the second sensor data to confirm the location of the user. Alternatively or in addition, the location prediction engine 122 uses the two types of sensor data to distinguish which of multiple candidate locations corresponds to the actual physical location of the user. For instance, the location prediction engine 122 detects at least two candidate locations of the user based on the first sensor data. Based on the second sensor data, the location prediction engine 122 selects one of the at least two candidate locations as the location of the user (or eliminates all but one of the candidate locations).

An activity that the user will perform at the location is predicted based on the location of the user (block 1506). By way of example, the recommendation engine 124 predicts an activity 414 that the user will perform at the location 408. Examples of activities include eating, exercising, and sleeping. Certainly, the system may predict other activities without departing from the spirit or scope of the described techniques.

A recommendation to control a glycemic response of the user to the predicted activity is generated (block 1508). By way of example, the recommendation engine 124 of the glycemic control system 110 generates a recommendation 422 to control a glycemic response 424 of the user to the predicted activity 414. In other words, the glycemic control system 110 uses the activity 414 the user is predicted to perform to generate one or more recommendations 422 for controlling a glycemic response 424 of the user, e.g., the person 102's glycemic response to the predicted activity. For example, the recommendation engine 124 generates recommendations 422 for one or more therapies to mitigate potential adverse effects due to the predicted activity 414. Examples of adverse effects include at least glucose excursions from a safe glucose range, such as hyperglycemia or hypoglycemia.

Having described examples of procedures in accordance with one or more implementations, consider now an example of a system and device that can be utilized to implement the various techniques described herein.

Example System and Device

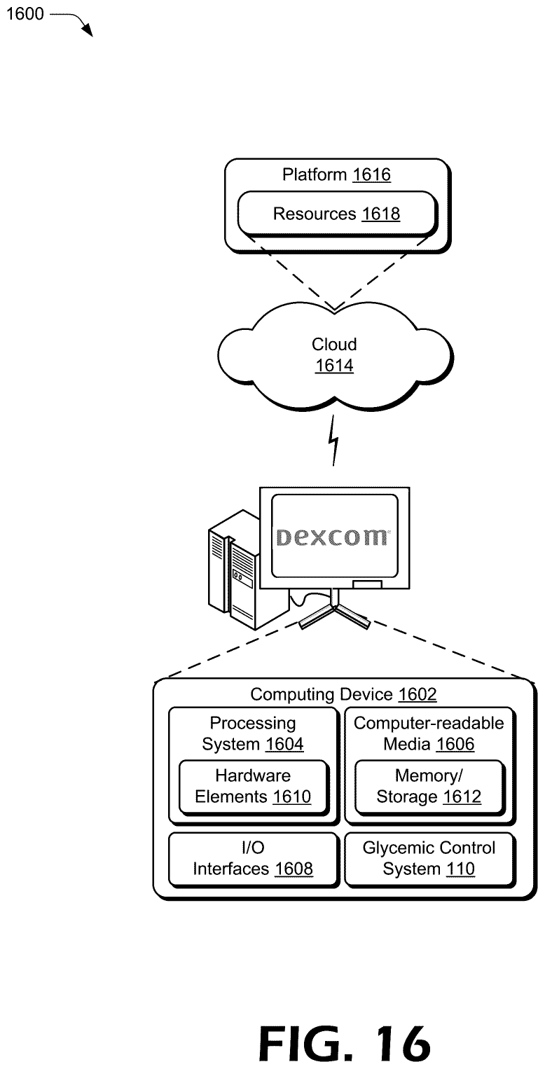

FIG. 16 illustrates an example of a system generally at 1600 that includes an example of a computing device 1602 that is representative of one or more computing systems and/or devices that may implement the various techniques described herein. This is illustrated through inclusion of the glycemic control system 110. The computing device 1602 may be, for example, a server of a service provider, a device associated with a client (e.g., a client device), an on-chip system, and/or any other suitable computing device or computing system.

The example computing device 1602 as illustrated includes a processing system 1604, one or more computer-readable media 1606, and one or more I/O interfaces 1608 that are communicably coupled, one to another. Although not shown, the computing device 1602 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1604 is representative of functionality to perform one or more operations using hardware. Accordingly, the processing system 1604 is illustrated as including hardware elements 1610 that may be configured as processors, functional blocks, and so forth. This may include implementation in hardware as an application specific integrated circuit or other logic device formed using one or more semiconductors. The hardware elements 1610 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 1606 is illustrated as including memory/storage 1612. The memory/storage 1612 represents memory/storage capacity associated with one or more computer-readable media. The memory/storage 1612 may include volatile media (such as random access memory (RAM)) and/or nonvolatile media (such as read only memory (ROM), Flash memory, optical disks, magnetic disks, and so forth). The memory/storage 1612 may include fixed media (e.g., RAM, ROM, a fixed hard drive, and so on) as well as removable media (e.g., Flash memory, a removable hard drive, an optical disc, and so forth). The computer-readable media 1606 may be configured in a variety of other ways as further described below.

Input/output interface(s) 1608 are representative of functionality to allow a user to enter commands and information to computing device 1602, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1602 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 1602. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1602, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1610 and computer-readable media 1606 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1610. The computing device 1602 may be configured to implement particular instructions and/or functions corresponding to the software and/or hardware modules. Accordingly, implementation of a module that is executable by the computing device 1602 as software may be achieved at least partially in hardware, e.g., through use of computer-readable storage media and/or hardware elements 1610 of the processing system 1604. The instructions and/or functions may be executable/operable by one or more articles of manufacture (for example, one or more computing devices 1602 and/or processing systems 1604) to implement techniques, modules, and examples described herein.

The techniques described herein may be supported by various configurations of the computing device 1602 and are not limited to the specific examples of the techniques described herein. This functionality may also be implemented all or in part through use of a distributed system, such as over a “cloud” 1614 via a platform 1616 as described below.

The cloud 1614 includes and/or is representative of a platform 1616 for resources 1618. The platform 1616 abstracts underlying functionality of hardware (e.g., servers) and software resources of the cloud 1614. The resources 1618 may include applications and/or data that can be utilized while computer processing is executed on servers that are remote from the computing device 1602. Resources 1618 can also include services provided over the Internet and/or through a subscriber network, such as a cellular or Wi-Fi network.

The platform 1616 may abstract resources and functions to connect the computing device 1602 with other computing devices. The platform 1616 may also serve to abstract scaling of resources to provide a corresponding level of scale to encountered demand for the resources 1618 that are implemented via the platform 1616. Accordingly, in an interconnected device embodiment, implementation of functionality described herein may be distributed throughout the system 1600. For example, the functionality may be implemented in part on the computing device 1602 as well as via the platform 1616 that abstracts the functionality of the cloud 1614.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method comprising:
obtaining sensor data from one or more sensors;
detecting, based on the sensor data, a location of a user;
predicting, based on the location of the user, an activity that the user will perform at the location; and
generating a recommendation to control a glycemic response of the user to the predicted activity.

2. The method of claim 1, wherein the generating the recommendation further comprises:
predicting the glycemic response of the user to the predicted activity;
determining whether the glycemic response of the user to the predicted activity will cause an adverse health event; and
responsive to determining that the glycemic response of the user to the predicted activity will cause the adverse health event, determining a mitigating therapy to prevent the adverse health event, wherein the recommendation includes the mitigating therapy.

3. The method of claim 1, wherein the detecting the location of the user is based on at least two different types of sensor data.

4. The method of claim 1, wherein the detecting the location of the user comprises detecting the location of the user based on first sensor data, and confirming the location of the user based on second sensor data.

5. The method of claim 1, wherein the detecting the location of the user comprises:
detecting at least two candidate locations of the user based on first sensor data; and
selecting one of the at least two candidate locations as the location of the user based on second sensor data.

6. The method of claim 1, wherein the recommendation comprises administering an amount of a medicament to the user to control the glycemic response of the user to the predicted activity.

7. The method of claim 6, further comprising communicating instructions to a medicament delivery system, the instructions causing the medicament delivery system to administer the amount of the medicament to the user.

8. The method of claim 6, wherein the medicament comprises insulin.

9. The method of claim 1, wherein the predicting the activity comprises predicting, based on the location, a state of the activity, the state comprising a pre-activity state, an activity state, or a post-activity state, wherein the recommendation is based on the state of the activity.

10. The method of claim 9, further comprising:
receiving additional sensor data;
determining, based on the additional sensor data, an additional location of the user;
predicting, based on the additional location of the user, a different state of the activity; and
generating an additional recommendation based on the different state of the activity.

11. The method of claim 1, further comprising causing display of the recommendation to control the glycemic response of the user on a computing device.

12. The method of claim 1, wherein the predicted activity comprises exercising, eating, or sleeping.

13. A system comprising:
one or more sensors to obtain sensor data associated with a user;
a medicament delivery system to administer a medicament to the user; and
at least a memory and a processor to perform operations comprising:
detecting, based on the sensor data, a location of the user;
predicting, based on the location of the user, an activity that the user will perform at the location;
determining an amount of the medicament to administer to the user to control a glycemic response of the user to the predicted activity; and
communicating instructions to the medicament delivery system, the instructions causing the medicament delivery system to deliver the amount of the medicament to the user.

14. The system of claim 13, further comprising an analyte monitoring device to obtain analyte data of the user, wherein the analyte monitoring device and the medicament delivery system are connected in a closed loop.

15. The system of claim 14, wherein the amount of the medicament is determined based at least in part on analyte data of the user obtained from the analyte monitoring device.

16. The system of claim 13, wherein the instructions cause the medicament delivery system to deliver the amount of the medicament to the user without user input.

17. The system of claim 13, wherein the medicament comprises insulin, and wherein the medicament delivery system comprises an insulin pump.

18. A method comprising:
detecting, based on at least two different types of sensor data, a location of a user;
predicting, based on the location of the user, an activity that the user will perform at the location;
predicting a glycemic response of the user to the predicted activity;
determining whether the glycemic response of the user to the predicted activity will cause an adverse health event; and
responsive to determining that the glycemic response of the user to the predicted activity will cause the adverse health event, communicating instructions to an insulin pump to cause the insulin pump to control the glycemic response of the user by administering insulin to the user.

19. The method of claim 18, further comprising obtaining glucose measurements of the user from a glucose monitoring device worn by the user, wherein the predicting the glycemic response further comprises predicting the glycemic response of the user to the predicted activity based at least in part on the glucose measurements.

20. The method of claim 19, wherein the glucose monitoring device and the insulin pump are connected in a closed loop.
